(19) United States
(12) Patent Application Publication
(10) Pub. No.: US 2024/0415832 A1
(43) Pub. Date: Dec. 19, 2024

(54) NOVEL METHODS

(71) Applicant: INTRA-CELLULAR THERAPIES, INC., New York, NY (US)

(72) Inventors: Peng LI, New Milford, NJ (US); Allen A. FIENBERG, New York, NY (US); Gretchen SNYDER, New York, NY (US); Robert E. DAVIS, San Diego, CA (US)

(21) Appl. No.: 18/703,145

(22) PCT Filed: Oct. 14, 2022
(86) PCT No.: PCT/US22/78177

§ 371 Date: Apr. 19, 2024

Related U.S. Application Data
(60) Provisional application No. 63/262,732, filed on Oct. 19, 2021.

(51) Int. Cl. A61K 31/4985; A61P 25/36

(52) U.S. Cl. CPC A61K 31/4985; A61P 25/36

(57) ABSTRACT

The invention relates to particular substituted heterocycle fused gamma-carbolines, in free, solid, pharmaceutically acceptable salt and/or substantially pure form as described herein, pharmaceutical compositions thereof, for use in methods for the treatment for the treatment and/or prevention of fentanyl-analog induced overdose and related sequelae.

### CROSS-REFERENCE TO RELATED APPLICATIONS

This international application claims priority to, and the benefit of, U.S. Provisional Application Ser. No. 63/262,732, filed on Oct. 19, 2021, the contents of which are hereby incorporated by reference in its entirety.

### FIELD OF THE INVENTION

The invention relates to the use of particular substituted heterocycle fused gamma-carbolines, in free or pharmaceutically acceptable salt and/or substantially pure form as described herein, pharmaceutical compositions thereof, for the treatment and/or prevention of fentanyl-analog induced overdose and related sequelae.

### BACKGROUND OF THE INVENTION

Substituted heterocycle fused gamma-carbolines are known to be agonists or antagonists of 5-HT₂ receptors, particularly 5-HT_{2A} receptors, in treating central nervous system disorders. These compounds have been disclosed, for example, in U.S. Pat. Nos. 6,552,017; 7,183,282; and RE39680, as novel compounds useful for the treatment of disorders associated with 5-HT_{2A} receptor modulation such as anxiety, depression, and psychosis. U.S. Pat. No. 7,081,455 discloses other gamma-carbolines as serotonin agonists and antagonists useful for the control and prevention of central nervous system disorders such as addictive behavior and sleep disorders. U.S. Pat. No. 8,598,119 discloses use of particular substituted heterocycle fused gamma-carbolines for the treatment of a combination of psychosis and depressive disorders as well as sleep, depressive and/or mood disorders in patients with psychosis or Parkinson's disease. U.S. Pat. No. 8,309,722 discloses methods of making substituted heterocycle fused gamma-carbolines. U.S. Pat. No. 8,648,077 also discloses methods of preparing toluenesulfonic acid addition salt crystals of these substituted heterocycle fused gamma-carbolines.

In addition, US 2021/00600009, incorporated herein by reference, discloses that some of the aforementioned substituted fused heterocycle gamma carbolines may operate, in part, through NMDA receptor antagonism via mTOR1 signaling, in a manner similar to that of ketamine. Ketamine is a selective NMDA receptor antagonist. Ketamine acts through a system that is unrelated to the common psychogenic monoamines (serotonin, norepinephrine and dopamine), and this is a major reason for its much more rapid effects. Ketamine directly antagonizes extrasynaptic glutamatergic NMDA receptors, which also indirectly results in activation of AMPA-type glutamate receptors. The downstream effects involve the brain-derived neurotrophic factor (BDNF) and mTORC1 kinase pathways. Similar to ketamine, recent evidence suggests that compounds related to those of the present disclosure enhance both NMDA and AMPA-induced currents in rat medial prefrontal cortex pyramidal neurons via activation of D1 receptors, and that this is associated with increased mTORC1 signaling.

U.S. Pat. Nos. 10,245,260, and 10,799,500, disclose additional novel fused heterocycle gamma carbolines, which, in addition to providing serotonin receptor inhibition, SERT inhibition, and dopamine receptor modulation, were also unexpectedly found to show significant activity at mu-opioid receptors. Analogs of these novel compounds have also been disclosed, for example, in publications U.S. Pat. Nos. 10,961,245 and 10,906,906, and 11,376,249, and 11,427,587, and in publication US 2021/0163481, the contents of each of which are incorporated by reference in their entireties. Among the indications disclosed in these publications are, generally, the treatment of pain, neuropathic pain, and chronic pain. Additional treatment indications for these compounds are disclosed in US 2021/0145829, US 2021/0093634, and in WO 2021/206391 (US 2022/0184072), the contents of each of which are incorporated by reference in their entireties. Synthetic methods for such compounds are also disclosed in WO 2020/131895 (US 2022/0041600), the contents of which are hereby incorporated by reference in its entirety.

For example, the Compound of Formula A, shown below, is a potent serotonin 5-HT_{2A} receptor antagonist and mu-opioid receptor partial, biased agonist. This compound also interacts with dopamine receptors, in particular dopamine D1 receptors.

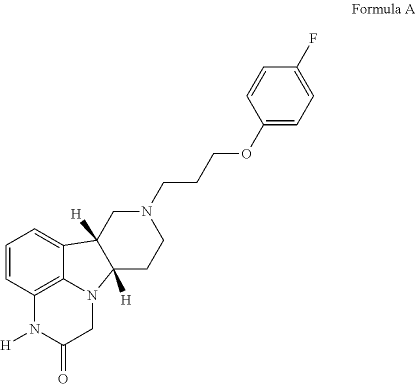 [SMILES: O=C1CN2c3c(cccc3[C@@H]3CN(CCCOc4ccc(F)cc4)CC[C@@H]32)N1]

It is also believed that the Compound of Formula A, via its D1 receptor activity, may also enhance NMDA and AMPA mediated signaling through the mTOR pathway. The Compound of Formula A is thus useful for the treatment or prophylaxis of central nervous system disorders, including opioid addiction, such as opioid use disorder, and for the treatment of pain disorders, such as chronic pain and neuropathic pain.

The compound of Formula A, and related compounds, are particularly useful because of their property of biased mu-opioid receptor activity. Depending on the cell type, or even within the same cell type, the intracellular domain of an activated mu opioid receptor can interact either with inhibitory G proteins or with beta-arrestin. The binding of a non-biased agonist to the mu-opioid receptor will result in approximately equal activation of both G-protein signaling and beta-arrestin signaling.

In contrast, when a biased agonist binds to a mu opioid receptor, it binds in such a way as to bias the intracellular domain of the receptor to interact with the G protein instead of the beta-arrestin. Thus, the compound of Formula A, and related compounds, act as partial or full agonists of the mu-opioid receptor's G-protein coupled signaling, but as an antagonist of the receptor's beta-arrestin signaling. This is in contrast to traditional opioid agonists, such as morphine and fentanyl, which tend to strongly activate both G-protein signaling and beta-arrestin signaling pathways. The activation of beta-arrestin signaling by such drugs is thought to mediate the gastrointestinal dysfunction, addiction, and respiratory depression effects typically mediated by opioid drugs, while the analgesic and anesthetic effects of mu-opioid receptor agonists are mediated by the G-protein signaling pathway.

This same effect has also been shown in pre-clinical studies and Phase II and Phase III clinical trials of the biased mu-receptor agonist oliceridine. Oliceridine has been shown to result in biased mu-opioid receptor agonism via G-protein coupled signaling with reduced beta-arrestin signaling compared to morphine, and this has been linked to its ability to produce analgesia with reduced respiratory side effects compared to morphine.

Furthermore, because biased agonists antagonize the beta-arrestin pathway, they are known to be generally useful in treating opioid overdose—by reversing the respiratory depression caused by the opioid. Beneficially, however, they will do so while still providing pain relief Biased beta-arrestin antagonists are expected to be useful in treating opioid overdose, because they will inhibit the most severe opioid adverse effects but still provide pain relief.

The United States is currently in the throes of a widespread opioid abuse epidemic that began in the late 1990's and is fueled by a combination of overprescribed prescription opioids (such as oxycodone, sold as OxyContin by Purdue Pharma), cheap imported illicit heroin, and a combination of licit and illicit fentanyl. While heroin and oxycodone (along with codeine, hydrocodone, hydromorphone, oxymorphone, and several other drugs) are natural or semi-synthetic analogs of morphine, fentanyl was the first and most prominent of a newer class of synthetic opioids. Unlike the natural and semi-synthetic opioids, fentanyl and fentanyl analogs do not have the complete classic pentacyclic core skeleton of morphine. Instead, fentanyl and fentanyl analogs share a common 4-aminophenyl(piperidine) core. The most common fentanyl analogs are sufentanil, alfentanil, remifentanil, and carfentanil:

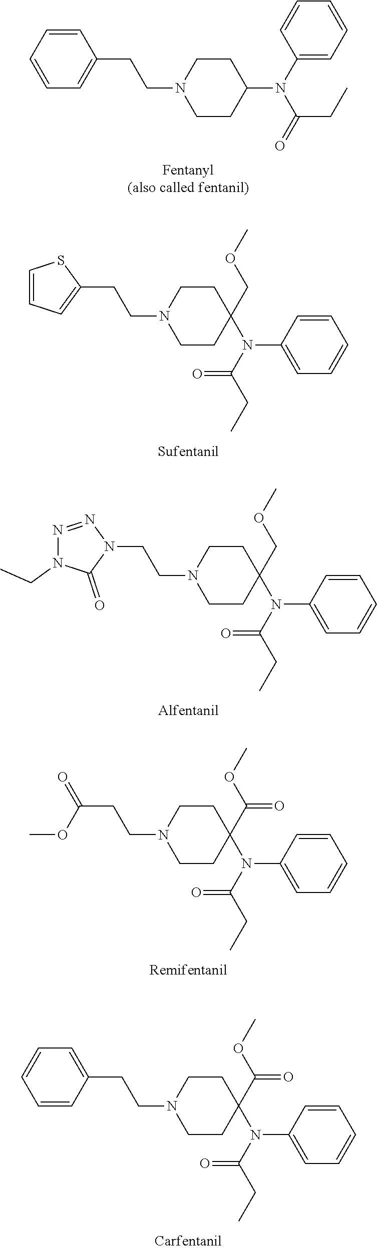 [SMILES: CCC(=O)N(c1ccccc1)C1CCN(CCc2ccccc2)CC1 ; CCC(=O)N(c1ccccc1)C1(COC)CCN(CCc2cccs2)CC1 ; CCC(=O)N(c1ccccc1)C1(COC)CCN(CCn2nnn(CC)c2=O)CC1 ; CCC(=O)N(c1ccccc1)C1(C(=O)OC)CCN(CCC(=O)OC)CC1 ; CCC(=O)N(c1ccccc1)C1(C(=O)OC)CCN(CCc2ccccc2)CC1]

Fentanyl and its analogs are substantially more potent than both morphine and heroin, due to either stronger mu-opioid receptor binding, higher lipophilicity, or both. The higher lipophilicity of these drugs, compared to morphine and heroin, results in them crossing the blood-brain barrier much faster, so that even with comparable receptor binding they are more potent. Fentanyl is generally considered about 50 times more potent than heroin and 100 times more potent than morphine (some sources indicate it as 150 times more potent than morphine). Sufentanil is considered 5 to 10 times more potent than fentanyl, and carfentanil about 100 times more potent than fentanyl (and thus 10,000 times more potent than morphine).

Because of its extremely high potency, and widespread cheap availability, it has become increasingly common for amphetamines, heroin and other street drugs to be adulterated with varying, and unpredictable, amounts of fentanyl. As a result of this and other trends, fentanyl has become a leading cause of opioid overdose in the United States, and especially, of opioid-related deaths. By 2016, fentanyl was the cause of at least 50% of opioid deaths, rising to more than 70% of deaths in 2017 and 2018. See Torralva & Janowsky, J. Pharmacol. Exp. Ther. 371:453-475 (2019). In fact, the rate of amphetamine overdoses has increased substantially in the last few years, driven primarily by the adulteration of amphetamine with fentanyl. In only a 5-year period, there was a 4-fold rise in amphetamine mortality, primarily linked to fentanyl adulteration.

Only three fentanyl analogs are approved for human use (sufentanil, alfentanil, remifentanil) while one is approved for veterinary use only (carfentanil). Nevertheless, these and a host of other novel synthetic fentanyl analogs have been found as adulterants in numerous street drugs, including amphetamines, heroin, cocaine, alprazolam (Xanax) and hydrocodone/paracetamol (Norco). See Armenian et al., Neuropharmacology (2017). Until 2013, there were only sporadic outbreaks of fentanyl or fentanyl analogs contaminating the U.S. heroin supply, but since then, such compounds have widely infiltrated North America, contaminating both heroin and cocaine. Deaths from fentanyl-laced heroin and cocaine doubled from 2012 to 2014. Street-purchased counterfeit Xanax and Norco caused two outbreaks in California in 2015-2016. The adulteration of non-opioid drugs with fentanyl and fentanyl analogs is particularly concerning because the users of such drugs are likely to be opioid naïve (thus having little or no established drug tolerance), and thus have significantly worse clinical outcomes. As testing for standard fentanyl analogs became more widespread (both in the medical setting and the forensic setting), illicit manufacturers began switching to novel synthetic fentanyl derivatives to evade detection, and today, numerous such illicit compounds are known and available on the black market from manufacturers in China and elsewhere. At least 21 synthetic opioid compounds are scheduled by the U.S. Drug Enforcement Administration today.

Because of its high potency and high lipophilicity, fentanyl-induced overdose is much more difficult to treat than morphine, heroin or oxycodone overdose. Fentanyl has an extraordinarily rapid onset of action, which makes reversal via mu-receptor antagonist (e.g., naloxone or naltrexone) treatment difficult in the outpatient setting (response time for EMS or police often being longer than the time for severe respiratory depression to develop). Larger doses of mu-receptor antagonists are also required to reverse fentanyl overdose, and there are limits on the rate and dose of mu-opioid antagonists that can be safely administered. While morphine takes an average of 19 minutes to reach 80% of peak effect, fentanyl produces severe respiratory depression much more rapidly.

Even more worrying, however, is that fentanyl and its analogs have an additional mechanism of action that has become extremely important in the ongoing opioid epidemic. While all opioids cause respiratory depression via mu-opioid receptor activation of the beta-arrestin signaling pathway in the brain, for reasons that are not yet entirely clear, fentanyl and its analogs can also cause a rapid combination of vocal cord closure (laryngospasm) and severe muscle rigidity in the chest wall and diaphragm. This can result from intravenous, transdermal, or inhalational administration of fentanyl and fentanyl analogs. Neither morphine, heroin, nor any other opioids having the classic morphine skeleton have this property. This severe chest wall rigidity has been referred to as fentanyl-induced respiratory muscle rigidity (FIRMR) (or simply fentanyl-induced muscle rigidity FIMR), and the combination of FIRMR and laryngospasm is clinically known as wooden chest syndrome (WCS). WCS can develop within only 1-2 minutes of injection of fentanyl, fentanyl analogs, or heroin or other drugs laced with fentanyl or its analogs. WCS has been demonstrated following as little as 50 micrograms of intravenous fentanyl.

The primary cause of mortality in WCS appears to be from the mechanical disruption of ventilation caused by closure of the glottic structures and upper airway. Laryngospasm is defined as the involuntary closure or occlusion of the glottic opening, and this is controlled by the intrinsic muscles of the larynx. These muscles are innervated by both sympathetic (adrenergic) and parasympathetic (cholinergic) nerve fibers, and the ultimate activity of these muscles is thus determined by the balance of sympathetic and parasympathetic input.

While FIRMR and WCS have long been known in the surgical anesthetic community (because it commonly occurs within the therapeutic dose range for surgical anesthesia), these conditions are not well-known in the first responder or emergency medical communities. This often leads to rapid death of drug abusers because those treating them are not aware of these effects of fentanyl (often compounded also by the lack of the patient's knowledge of having taken something having fentanyl in it). Numerous eyewitness and survivor accounts of overdoses report a very rapid onset of cyanosis, loss of consciousness, severe muscle rigidity, and seizure like behavior, immediately following injection of drug. The rapid onset of death is very unlike the respiratory depression normally associated with morphine, heroin, and oxycodone overdoses. Indeed, mechanical failure of respiration in a fentanyl or fentanyl analog overdose usually develops less than 2 minutes after drug administration and presents prior to centrally-mediated respiratory depression (50% drop in respiratory mechanics takes 7-9 minutes to develop).

Even more worrying, the standard first-line therapies for opioid overdose—naloxone, naltrexone, and nalmefene—are not effective in reversing these fentanyl-induced effects. The severe chest wall rigidity also compromises the effectiveness of chest compressions in cardiopulmonary resuscitation. As a result, while the ratio of emergency room visits to death for heroin-related overdose has been reported as about 10:1, the ratio is only 1:1 for fentanyl-related overdoses.

The standard dose of intravenous naloxone administered for opioid overdose is 0.4 to 2 mg, with additional doses at 2-to-3-minute intervals, up to a maximum of 10 mg. However, intranasal naloxone, which is widely used by first responders, is recommended for only a 4 mg maximum total dose. See, e.g., Williams et al., Prehospital Emergency Care 23(6):749-63 (2019). In one study, however, it was found that while the upper airway effects of morphine could be fully blocked by a dose of 0.1 mg/kg of naloxone (e.g., 7 mg for a 70-kg person), to fully block the upper airway effects of fentanyl required from 0.8 to 1.6 mg/kg of naloxone (56 to 112 mg for a 70-kg person). A study examining a 2006 fentanyl overdose outbreak reported that 0.4 to 12 mg of naloxone was administered to patients in a hospital emergency room, with only 15% patients responding to a 0.4 mg dose, and 6 patients out of 26 requiring at least 6 mg to reverse respiratory depression. In another study examining 18 patients who overdosed on counterfeit hydrocodone/paracetamol contaminated with fentanyl, 0.4 to 8 mg intravenous bolus injections of naloxone were required, and 4 of the patients required naloxone infusions lasting 26-40 hours.

Unfortunately, however, high doses of naloxone are not practical for therapeutic use because the rapid injection of as little as 0.4 mg of naloxone (0.0057 mg/kg for a 70 kg adult) in active opioid users commonly results in laryngospasm, pulmonary edema, hemodynamic instability, and cardiac arrythmia (all due to catecholamine release). High-dose naloxone treatment is therefore contraindicated, especially in the field. Thus, in the field-without additional medical and pharmacological support—it is normally quite difficult, if not impossible to, to use naloxone to reverse fentanyl-induced overdose before it becomes fatal.

It is clear that WCS is not simply the result of mu-opioid receptor agonism-since other powerful mu-opioid agonists do not cause WCS (e.g., morphine), and since powerful mu-opioid antagonists (e.g., naloxone) do not reverse WCS at normal dose ranges. Thus, fentanyl and its analogs must cause WCS by some other mechanism which involves other neurotransmitter systems.

There is evidence, both from in vitro studies and from various animal models, which indicates that fentanyl exerts these effects via the stimulation of noradrenergic activity, and possibly cholinergic activity, in the locus coeruleus (LC) region of the brain. Without being bound by theory, it is believed that in the LC, fentanyl acts as an agonist of mu-opioid receptors, and the resulting hyperpolarization of the LC neuron results in efferent noradrenergic neuron activity, specifically, in coerulospinal fibers connected to spinal motor neurons terminating in the chest wall and abdomen, as well as laryngeal nerve fibers contributing to the vagal nerve via the superior cervical and middle cervical ganglia. These laryngeal nerve fibers directly innervate the intrinsic muscles of the larynx.

The role of the alpha1-adrenergic receptor, in particular, has been indicated by animal experiments demonstrating that the selective alpha1-adrenergic antagonist prazosin, administered intravenously ten minutes prior to fentanyl, inhibits the development of FIMR, and the same result occurs with ablation of the LC region of the brain. Other studies show that intrathecal administration of prazosin at the L3 spinal level also inhibits FIMR, but the administration of yohimbine, an alpha2-adrenergic antagonist, does not. There has also been some animal evidence that fentanyl itself is an antagonist of the alpha1-adrenergic receptor, although weakly, and with selectivity for the alpha1B and alpha1A receptors (rather than the alpha1D receptor). Unfortunately, these studies do not directly predict the beneficial use of alpha1-adrenergic antagonists in treating opioid overdose, because the corresponding human doses used in the animal studies would result in lethal hypotension in humans.

There is also increasing evidence for an intermediate role for GABA interneurons in the pathogenesis of WCS. GABA interneurons are part of an inhibitory network throughout the brain, and they are particularly abundant in the LC. The LC is responsible for maintaining basal skeletal muscle tone in the torso via the noradrenergic activation of spinal motor neurons, but norepinephrine release from the LC presynaptic terminals is inhibited by the GABA efferent signaling. Inhibition of the GABA interneurons, therefore, results in increased skeletal muscle tone via increased LC noradrenergic activity. Without being bound by theory, it is believed that fentanyl binds to mu-opioid receptors on GABA interneurons, and that this results in inhibition of GABA interneuron afferents, resulting in release of the inhibition on LC sympathetic neurons.

There is also evidence that LC neurons are also high in muscarinic and nicotinic acetylcholine receptors. It is believed that as the LC receives cholinergic input from other brain regions, such as the pontine reticular formation, fentanyl-induced mu-receptor agonism in these neighboring regions may stimulate acetylcholine release, which results in further stimulation of norepinephrine release by the LC. There is also some evidence that fentanyl acts directly as an M3 muscarinic receptor antagonist, which may result in inhibition of parasympathetic tone at the laryngeal intrinsic muscles, further increasing the spasm resulting from sympathetic activation of these muscles.

NMDA and non-NMDA glutamate receptor activity has also been implicated in the pathogenesis of WCS.

Because of the intermediate role of these other neurotransmitters (e.g., norepinephrine, acetylcholine, GABA, etc.) in the pathogenesis of WCS, a further reason for the failure of response of WCS to mu-opioid antagonist treatment might be that once these indirect fentanyl-stimulated effects are initiated (by mu-receptor agonism), mere mu-receptor antagonism cannot reverse the effects already set in motion.

Finally, there is also evidence that fentanyl, but not morphine, has some activity as a norepinephrine reuptake inhibitor. It has been shown in various neural cell lines that this effect is not antagonized by naloxone, indicating that it is not an indirect effect of mu-receptor agonism. Thus, it is also possible that fentanyl is exerting a direct effect on neurons in the LC and stimulating hyperactivity of the muscles involved in FIMR and WCS.

Thus, there remains a need for therapeutic agents which are particularly suited to reversing the effects of an acute fentanyl overdose.

### SUMMARY OF THE INVENTION

The present disclosure relates to the treatment of disorders associated with the abuse of, and overdose with, fentanyl and fentanyl analogs. Fentanyl analogs include, but are not limited to, the compounds sufentanil, alfentanil, remifentanil, carfentanil, as well as derivatives of these compounds, as further explained herein. Fentanyl and fentanyl analogs are collectively referred to herein as “F/FA.”

The present disclosure provides a method for one or more of the following:
(a) treating or reversing F/FA overdose;
(b) treating or reversing F/FA-induced respiratory depression;
(c) treating or reversing F/FA-induced muscle rigidity;
(d) treating or reversing F/A-induced laryngospasm;
(e) reversing or inhibiting binding of F/FA to mu-opioid receptors in the central nervous system (e.g., in the locus coeruleus);
(f) inhibiting F/FA-induced beta-arrestin signaling in the central nervous system (e.g., in the locus coeruleus);
(g) preventing death from F/FA overdose; and
(h) anesthetic recovery (e.g., following surgery);
   the method comprising administering to a patient in need thereof an effective amount of a Compound of Formula I, or a pharmaceutical composition thereof, wherein the Compound of Formula I is:

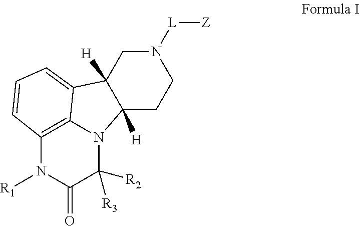

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸
R² and R³ are independently selected from H, D, C₁₋₆alkyl (e.g., methyl), C₁₋₆alkoxy (e.g., methoxy), halo (e.g., F), cyano, or hydroxy;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, or hydroxy;
Z is selected from aryl (e.g., phenyl) and heteroaryl (e.g., pyridyl, indazolyl, benzimidazolyl, benzisoxazolyl), wherein said aryl or heteroaryl is optionally substituted with one or more R⁴ moieties;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

In additional aspects, the present disclosure further provides use of a Compound of the present disclosure, e.g., a Compound of Formula I, in the manufacture of a medicament for the methods disclosed herein. The present disclosure further provides a Compound of the present disclosure, e.g., a Compound of Formula I, for use in the methods disclosed herein.

### BRIEF DESCRIPTION OF THE DRAWINGS

FIG. 1. Dose-response curve for inhibition of fentanyl-induced beta-arresting signaling of the mu-opioid receptor by the Compound of Example 1.

### DETAILED DESCRIPTION OF THE INVENTION

In a first aspect, the present disclosure provides a method for one or more of the following:
(a) treating or reversing F/FA overdose;
(b) treating or reversing F/FA-induced respiratory depression;
(c) treating or reversing F/FA-induced muscle rigidity;
(d) treating or reversing F/A-induced laryngospasm;
(e) reversing or inhibiting binding of F/FA to mu-opioid receptors in the central nervous system (e.g., in the locus coeruleus);
(f) inhibiting F/FA-induced beta-arrestin signaling in the central nervous system (e.g., in the locus coeruleus);
(g) preventing death from F/FA overdose; and
(h) anesthetic recovery (e.g., following surgery);
   the method comprising administering to a patient in need thereof an effective amount of a Compound of Formula I, or a pharmaceutical composition comprising a Compound of Formula I, wherein the Compound of Formula I is:

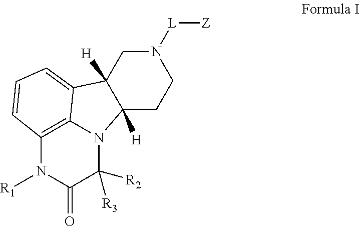

wherein:
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸
R² and R³ are independently selected from H, D, C₁₋₆alkyl (e.g., methyl), C₁₋₆alkoxy (e.g., methoxy), halo (e.g., F), cyano, or hydroxy;
L is C₁₋₆alkylene (e.g., ethylene, propylene, or butylene), C₁₋₆alkoxy (e.g., propoxy or butoxy), C₂₋₃alkoxyC₁₋₃alkylene (e.g., CH₂CH₂OCH₂), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, or hydroxy;
Z is selected from aryl (e.g., phenyl) and heteroaryl (e.g., pyridyl, indazolyl, benzimidazolyl, benzisoxazolyl), wherein said aryl or heteroaryl is optionally substituted with one or more R⁴ moieties;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

The present disclosure provides additional exemplary embodiments Method 1, including:
1.1 Method 1, wherein in the compound of Formula I, R¹ is H;
1.2 Method 1, wherein in the compound of Formula I, R¹ is C₁₋₆alkyl, e.g., methyl;
1.3 Method 1, wherein in the compound of Formula I, R¹ is —C(O)—O—C(R^{a})(R^{b})(R^{c});
1.4 Method 1.3, wherein in the compound of Formula I, R^{a} is H and R^{b} and R^{c} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.5 Method 1.3, wherein in the compound of Formula I, R^{a} and R^{b} are H and R^{c} is C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.6 Method 1.3, wherein in the compound of Formula I, R^{a}, R^{b} and R^{c} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.7 Method 1.3, wherein in the compound of Formula I, R^{a}, R^{b} and R^{c} are each H;
1.8 Method 1.3, wherein in the compound of Formula I, R^{a} and R^{b} are H and R^{c} is C₁₀₋₁₄alkyl (e.g., R^{c} is CH₃(CH₂)₁₀ or CH₃(CH₂)₁₄);
1.9 Method 1, wherein in the compound of Formula I, R¹ is —C(O)—O—CH₂—O—C(R^{a})(R^{b})(R^{c});
1.10 Method 1.9, wherein in the compound of Formula I, R^{a} is H and R^{b} and R^{c} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.11 Method 1.9, wherein in the compound of Formula I, R^{a} and R^{b} are H and R^{c} is C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.12 Method 1.9, wherein in the compound of Formula I, R^{a}, R^{b} and R^{c} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.13 Method 1.9, wherein in the compound of Formula I, R^{a}, R^{b} and R^{c} are each H;
1.14 Method 1, wherein in the compound of Formula I, R¹ is —C(R⁶)(R⁷)—O—C(O)—R^{g}, and R⁸ is —C(R^{a})(R^{b})(R^{c});
1.15 Method 1, wherein in the compound of Formula I, R¹ is —C(R⁶)(R⁷)—O—C(O)—R⁸, and R⁸ is —O—C(R^{a})(R^{b})(R^{c});
1.16 Method 1.14 or 1.15, wherein in the compound of Formula I, R^{a} is H and R^{b} and R^{c} are each independently selected from C₁₋₂₄alkyl, e.g., C₁₋₂₀alkyl, C₅₋₂₀alkyl, C₉₋₁₈alkyl, C₁₀₋₁₆alkyl, or C₁₁alkyl, C₁₂alkyl, C₁₃alkyl, C₁₄alkyl, C₁₅alkyl or C₁₆alkyl;
1.17 Method 1.14 or 1.15, wherein in the compound of Formula I, R^{a} and R^{b} are H and R^{c} is C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.18 Method 1.14 or 1.15, wherein in the compound of Formula I, R^{a}, R^{b} and R^{c} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.19 Method 1.14 or 1.15, wherein in the compound of Formula I, R^{a}, R^{b} and R^{c} are each H;
1.20 Any of Methods 1.14-1.19, wherein in the compound of Formula I, R⁶ is H, and R⁷ is C₁₋₃alkyl (e.g., R⁷ is methyl or isopropyl), and R⁸ is C₁₀₋₁₄alkyl (e.g., R⁸ is CH₃(CH₂)₁₀ or CH₃(CH₂)₁₄);
1.21 Method 1, wherein in the compound of Formula I, R¹ is —C(R⁶)(R⁷)—O—C(O)—R⁸, and R⁸ is —N(R^{d})(R^{e});
1.22 Method 1.21, wherein in the compound of Formula I, R^{d} is H and R^{e} is independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.23 Method 1.21, wherein in the compound of Formula I, R^{d} and R^{e} are each independently selected from C₁₋₂₄alkyl, e.g., C₁₋₂₀alkyl, C₅₋₂₀alkyl, C₉₋₁₅alkyl, C₁₀₋₁₆alkyl, or C₁₁alkyl, C₁₂alkyl, C₁₃alkyl, C₁₄alkyl, C₁₅alkyl or C₁₆alkyl;
1.24 Method 1.21, wherein in the compound of Formula I, R^{d} and R^{e} are each H;
1.25 Any of Methods 1.14-1.24, wherein in the compound of Formula I, R⁶ is H and R⁷ is H;
1.26 Any of Methods 1.14-1.24, wherein in the compound of Formula I, R⁶ is C₁₋₆alkyl and R⁷ is C₁₋₆alkyl;
1.27 Any of Methods 1.14-1.24, wherein in the compound of Formula I, R⁶ is H and R⁷ is C₁₋₆alkyl;
1.28 Any of Methods 1.14-1.24, wherein in the compound of Formula I, R⁶ is H and R⁷ is carboxy;
1.29 Any of Methods 1.14-1.24, wherein in the compound of Formula I, R⁶ is H and R⁷ is C₁₋₆alkoxycarbonyl, e.g., ethoxycarbonyl or methoxycarbonyl;
1.30 Method 1, or any of 1.1-1.29, wherein in the compound of Formula I, R² and R³ are H;
1.31 Method 1, or any of 1.1-1.29, wherein in the compound of Formula I, R² is H and R³ is D;
1.32 Method 1, or any of 1.1-1.29, wherein in the compound of Formula I, R² and R³ are D;
1.33 Method 1, or any of 1.1-1.32, wherein in the compound of Formula I, L is C₁₋₆alkylene (e.g., ethylene, propylene, or butylene), C₁₋₆alkoxy (e.g., propoxy), C₂₋₃alkoxyC₁₋₃alkylene (e.g., CH₂CH₂OCH₂) C₁₋₆alkylamino (e.g., propylamino or N-methylpropylamino), or C₁₋₆alkylthio (e.g., —CH₂CH₂CH₂S—), optionally substituted with one or more R⁴ moieties;
1.34 Method 1.33, wherein in the compound of Formula I, L is unsubstituted C₁₋₆alkylene (e.g., ethylene, propylene, or butylene);
1.35 Method 1.33, wherein in the compound of Formula I, L is C₁₋₆alkylene (e.g., ethylene, propylene, or butylene), substituted with one or more R⁴ moieties;
1.36 Method 1.33, wherein in the compound of Formula I, L is unsubstituted C₁₋₆alkyoxy (e.g., propoxy or butoxy);
1.37 Method 1.33, wherein in the compound of Formula I, L is C₁₋₆alkoxy (e.g., propoxy or butoxy), substituted with one or more R⁴ moieties;
1.38 Method 1.33, wherein in the compound of Formula I, L is unsubstituted C₂₋₃alkoxyC₁₋₃alkylene (e.g., CH₂CH₂OCH₂);
1.39 Method 1.33, wherein in the compound of Formula I, L is C₂₋₃alkoxyC₁₋₃alkylene (e.g., CH₂CH₂OCH₂), substituted with one or more R⁴ moieties;
1.40 Method 1, or any of 1.1-1.39, wherein in the compound of Formula I, R¹, R² and R³ are each H;
1.41 Method 1, or any of 1.1-1.40, wherein in the compound of Formula I, L is —(CH₂)ₙ—X—, and wherein n is an integer selected from 2, 3, and 4, and X is selected from —O—, —S—, —NH—, —N(C₁₋₆alkyl)-, and CH₂;
1.42 Method 1.41, wherein in the compound of Formula I, L is —(CH₂)ₙ—X—, and wherein n is an integer selected from 2, 3, and 4, and X is —O—;
1.43 Method 1.41, wherein in the compound of Formula I, L is —(CH₂)ₙ—X—, and wherein n is 3, and X is selected from —O—, —S—, —NH— and —N(C₁₋₆alkyl)- (e.g., —N(CH₃)—);
1.44 Method 1.41, wherein in the compound of Formula I, L is —(CH₂)ₙ—X—, and wherein n is 3, and X is CH₂;
1.45 Method 1, or any of 1.1-1.44, wherein in the compound of Formula I, Z is aryl (e.g., phenyl), optionally substituted with one or more R⁴ moieties;
1.46 Method 1.45, wherein in the compound of Formula I, Z is aryl (e.g., phenyl), substituted with one or more R⁴ moieties;
1.47 Method 1.46, wherein in the compound of Formula I, Z is phenyl substituted with one, two, three or four R⁴ moieties;
1.48 Method 1.47, wherein in the compound of Formula I, the one, two three or four R⁴ moieties are independently selected from halo (e.g., fluoro, chloro, bromo or iodo) and cyano;
1.49 Method 1.46, wherein in the compound of Formula I, Z is phenyl substituted with one R⁴ moiety selected from halo (e.g., fluoro, chloro, bromo or iodo) and cyano (e.g., Z is 4-fluorophenyl, or 4-chlorophenyl, or 4-cyanophenyl);
1.50 Method 1.46, wherein in the compound of Formula I, Z is phenyl substituted with one fluoro (e.g., 2-fluorophenyl, 3-fluorophenyl or 4-fluorophenyl);
1.51 Method 1.46, wherein in the compound of Formula I, Z is 4-fluorophenyl;
1.52 Method I, or any of 1.1-1.44, wherein in the compound of Formula I, Z is heteroaryl (e.g., pyridyl, indazolyl, benzimidazolyl, benzisoxazolyl), optionally substituted with one or more R⁴ moieties;
1.53 Method 1.52, wherein in the compound of Formula I, said heteroaryl is a monocyclic 5-membered or 6-membered heteroaryl (e.g., pyridyl, pyrimidyl, pyrazinyl, thiophenyl, pyrrolyl, furanyl, imidazolyl, oxazolyl, isoxazolyl, thiazolyl);
1.54 Method 1.53, wherein in the compound of Formula I, said heteroaryl is selected from pyridyl, pyrimidinyl, pyrazinyl, and thiophenyl;
1.55 Method 1.52, wherein in the compound of Formula I, said heteroaryl is a bicyclic 9-membered or 10-membered heteroaryl (e.g., indolyl, isoindolyl, benzofuranyl, benzothiophenyl, indazolyl, benzimidazolyl, benzoxazolyl, benzisoxazolyl, benzothiazolyl, quinolinyl, isoquinolinyl, quinoxalinyl, quinazolinyl, benzodioxolyl, 2-oxo-tetrahydroquinolinyl);
1.56 Method 1.55, wherein in the compound of Formula I, said heteroaryl is selected from indazolyl, benzisoxazolyl, quinolinyl, benzodioxolyl, and 2-oxo-tetrahydroquinolinyl;
1.57 Method 1.55, wherein in the compound of Formula I, said heteroaryl is selected from indazolyl, benzisoxazolyl, and quinolinyl;
1.58 Any of Methods 1.52-1.57, wherein in the compound of Formula I, said heteroaryl is substituted with one, two, three or four R⁴ moieties;
1.59 Method 1.58, wherein in the compound of Formula I, the one, two three or four R⁴ moieties are independently selected from halo (e.g., fluoro, chloro, bromo or iodo), cyano, hydroxy, or C₁₋₆alkoxy (e.g., methoxy);
1.60 Method 1.58 or 1.59, wherein in the compound of Formula I, said heteroaryl is substituted with one R⁴ moiety selected from halo (e.g., fluoro, chloro, bromo or iodo) and cyano (e.g., said heteroaryl is 6-fluoro-3-indazolyl, 6-chloro-3-indazolyl, 6-fluoro-3-benzisoxazolyl, or 5-chloro-3-benzisoxazolyl);
1.61 Method 1, or any of 1.1-1.60, wherein the compound of Formula I is selected from the group consisting of:

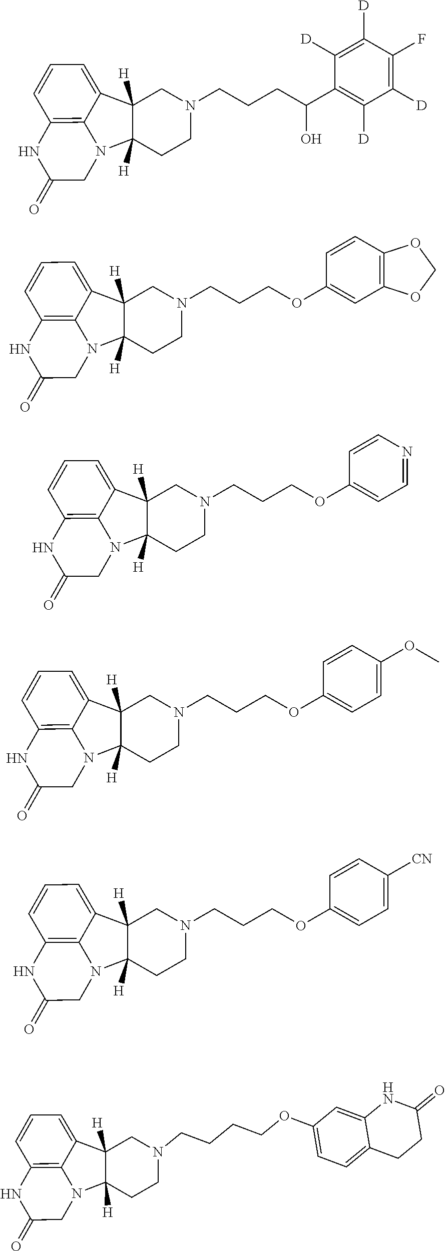 [SMILES: [2H]c1c([2H])c(C(O)CCCN2CC[C@H]3[C@@H](C2)c2cccc4c2N3CC(=O)N4)c([2H])c([2H])c1F ; O=C1CN2c3c(cccc3[C@@H]3CN(CCCOc4ccc5c(c4)OCO5)CC[C@@H]32)N1 ; O=C1CN2c3c(cccc3[C@@H]3CN(CCCOc4ccncc4)CC[C@@H]32)N1 ; COc1ccc(OCCCN2CC[C@H]3[C@@H](C2)c2cccc4c2N3CC(=O)N4)cc1 ; N#Cc1ccc(OCCCN2CC[C@H]3[C@@H](C2)c2cccc4c2N3CC(=O)N4)cc1 ; O=C1CCc2ccc(OCCCCN3CC[C@H]4[C@@H](C3)c3cccc5c3N4CC(=O)N5)cc2N1]

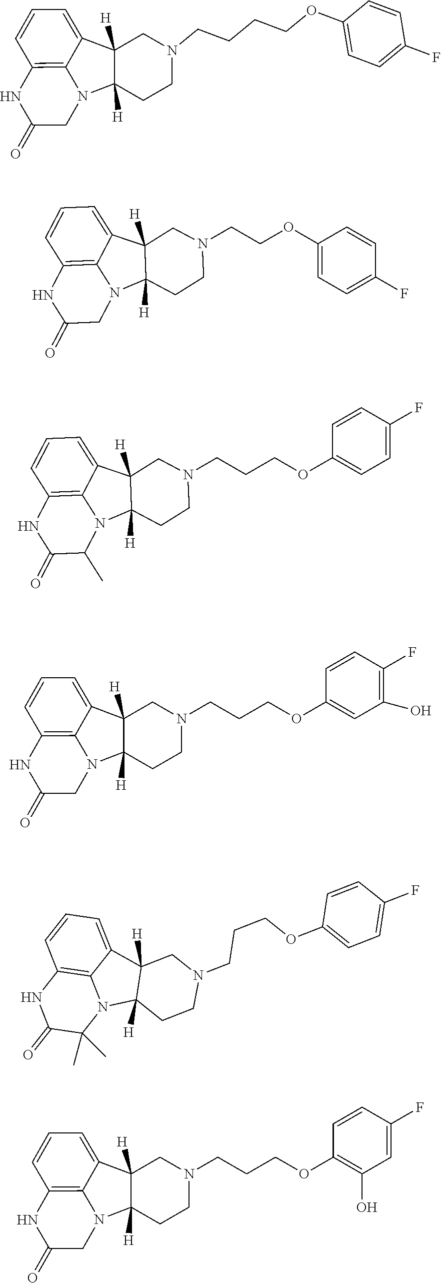 [SMILES: O=C1CN2c3c(cccc3[C@@H]3CN(CCCCOc4ccc(F)cc4)CC[C@@H]32)N1 ; O=C1CN2c3c(cccc3[C@@H]3CN(CCOc4ccc(F)cc4)CC[C@@H]32)N1 ; CC1C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)c(O)c4)CC[C@@H]32)N1 ; CC1(C)C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)cc4O)CC[C@@H]32)N1]

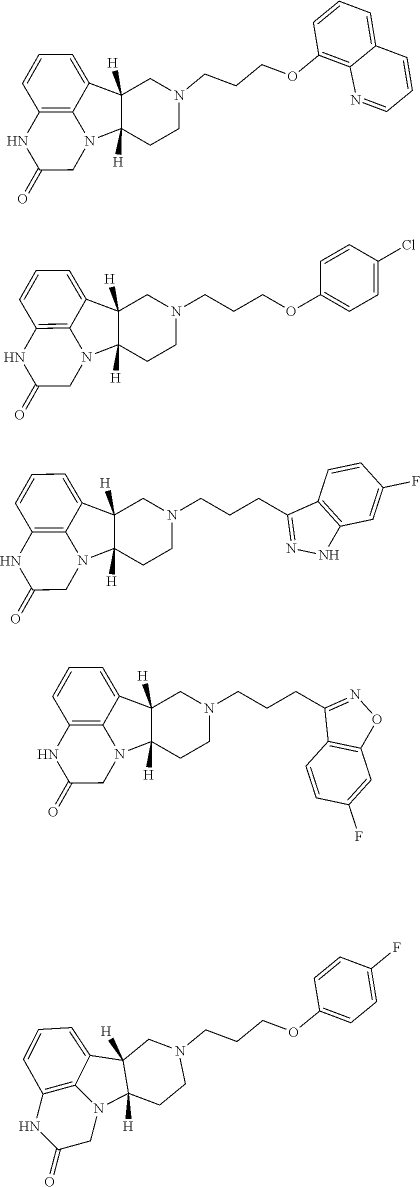 [SMILES: O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(Cl)cc4)CC[C@@H]32)N1 ; O=C1CN2c3c(cccc3[C@@H]3CN(CCCc4n[nH]c5cc(F)ccc45)CC[C@@H]32)N1 ; O=C1CN2c3c(cccc3[C@@H]3CN(CCCc4noc5cc(F)ccc45)CC[C@@H]32)N1 ; O=C1CN2c3c(cccc3[C@@H]3CN(CCCOc4ccc(F)cc4)CC[C@@H]32)N1]

each independently in free, or pharmaceutically acceptable salt form;
1.62 Method 1, or any of 1.1-1.60, wherein the compound of Formula I is selected from the group consisting of:

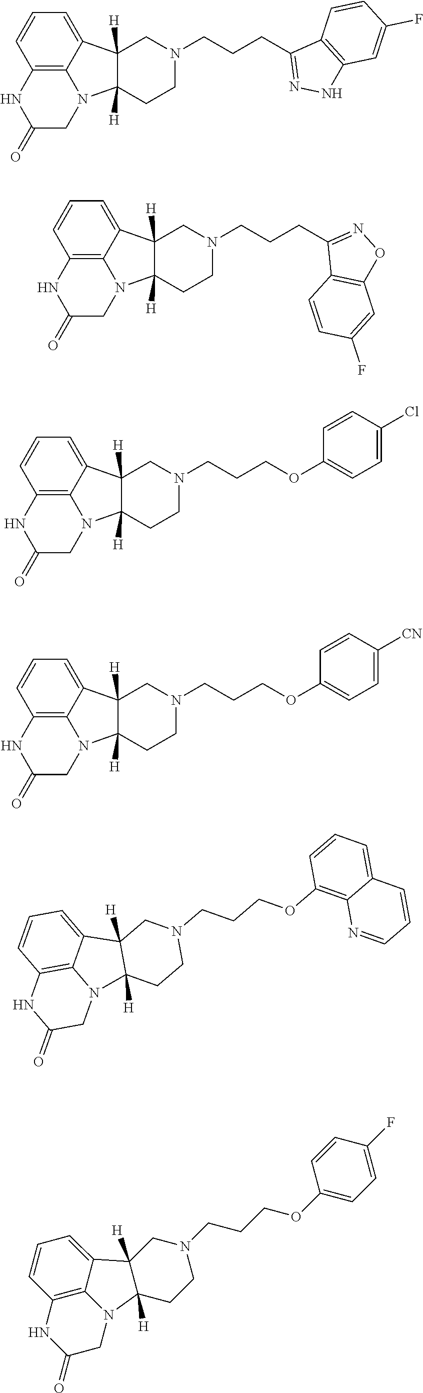

each independently in free or pharmaceutically acceptable salt form;
1.63 Method 1, or any of 1.1-1.60, wherein the compound of Formula I is selected from the group consisting of:

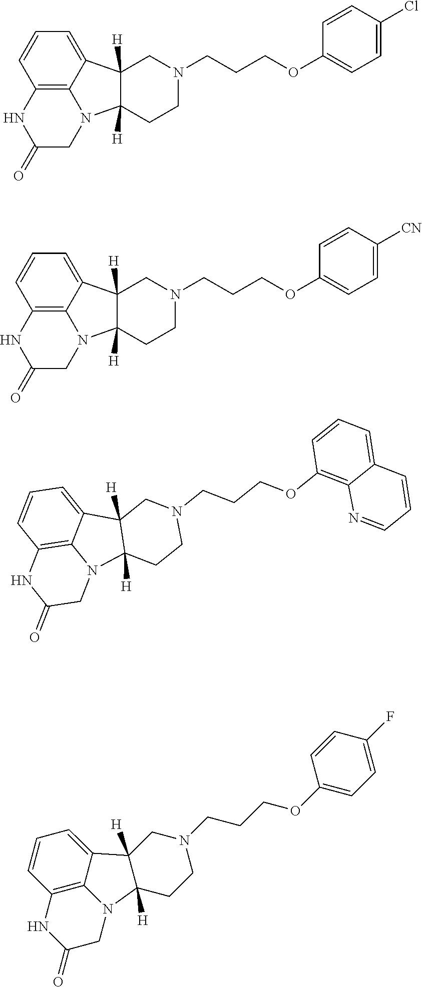 [SMILES: O=C1CN2c3c(cccc3[C@@H]3CN(CCCOc4ccc(Cl)cc4)CC[C@@H]32)N1 ; N#Cc1ccc(OCCCN2CC[C@H]3[C@@H](C2)c2cccc4c2N3CC(=O)N4)cc1 ; O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(F)cc4)CC[C@@H]32)N1]

each independently in free or pharmaceutically acceptable salt form;
1.64 Method 1, or any of 1.1-1.61, wherein the compound of Formula I is

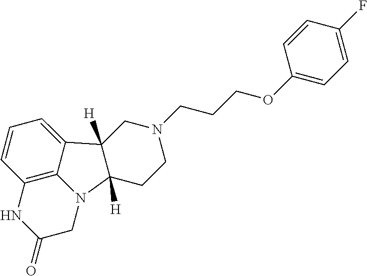 [SMILES: O=C1CN2c3c(cccc3[C@@H]3CN(CCCOc4ccc(F)cc4)CC[C@@H]32)N1]

in free or pharmaceutically acceptable salt form;
1.65 Method 1, or any of 1.1-1.64, wherein the compound of Formula I is in free form;
1.66 Method 1, or any of 1.1-1.64, wherein the compound of Formula I is in salt form, e.g., pharmaceutically acceptable salt form;
1.67 Method 1, or any of 1.1-1.64, wherein the compound of Formula I is in acid addition salt form, for example, wherein the acid is hydrochloric, toluenesulfonic, glutamic, tartaric, malic or ascorbic acid;
1.68 Method 1, or any of 1.1-1.67, wherein the compound of Formula I is in substantially pure diastereomeric form (i.e., substantially free from other diastereomers);
1.69 Method 1, or any of 1.1-1.67, wherein the compound of Formula I has a diastereomeric excess of greater than 70%, preferably greater than 80%, more preferably greater than 90% and most preferably greater than 95%;
1.70 Method 1, or any of 1.1-1.69, wherein the compound of Formula I is in solid form, e.g., in crystal form;
1.71 Method 1, or any of 1.1-1.70, wherein the compound of Formula I is in isolated or purified form (e.g., in at least 90% pure form, or at least 95% or at least 98% or at least 99%);
1.72 Method 1 or any of 1.1-1.71, wherein the compound of Formula I is administered in the form of a pharmaceutical composition comprising the compound of Formula I in admixture with a pharmaceutically acceptable diluent or carrier;
1.73 Method 1.72, wherein the compound of Formula I is in pharmaceutically acceptable salt form in admixture with a pharmaceutically acceptable diluent or carrier;
1.74 Method 1.72 or 1.73, wherein the pharmaceutical composition is an immediate-release formulation;
1.75 Any of Methods 1.72-1.74, wherein the pharmaceutical composition is formulated for single dose administration (e.g., tablets, capsules, wafers, single-use injections, single-use ampules or vials for intranasal administration, single use ampules or vials for injection, single use intranasal sprays);
1.76 Any of Methods 1.72-1.75, wherein the pharmaceutical composition is in the form of a tablet, capsule or wafer (e.g., an oral, sublingual, or buccal tablet, capsule, or wafer);
1.77 Method 1.76, wherein the pharmaceutical composition is a rapidly-dissolving oral tablet (e.g., a rapidly dissolving sublingual tablet);
1.78 Any of Methods 1.72-1.75, wherein the pharmaceutical composition is formulated for intranasal or intrapulmonary administration (e.g., as an aerosol, mist, or powder for inhalation);
1.79 Any of Methods 1.72-1.75, wherein the pharmaceutical composition is formulated for administration by injection, for example, as a sterile aqueous solution, e.g., for intravenous, subcutaneous, or intramuscular injection;
1.80 Method 1.79, wherein the pharmaceutical composition is formulated for intravenous, intrathecal, intramuscular, subcutaneous or intraperitoneal injection.
1.81 Any of Methods 1.72 to 1.80, wherein the pharmaceutical composition is formulated for and/or packaged as a pre-filled syringe for injection, as an auto-injector, or as a sterile solution in a vial for injection or intranasal administration.
1.82 Any of Methods 1.72 to 1.81, wherein the pharmaceutical composition is packaged as a kit for administration by nurses, emergency medical technicians or paramedics.
1.83 Any of Methods 1.72 to 1.81, wherein the pharmaceutical composition is packaged as a kit for administration by non-medical first responders (e.g., police officers or fire officers).
1.84 Any of Methods 1.72 to 1.81, wherein the pharmaceutical composition is packaged as a kit for administration by the general public (e.g., a single-use take-home kit, such as for drug abusers, family members and friends of drug-abusers, and for public venues, such as places of worship, community centers, sporting event venues).

As used herein, the term “Compound of the present disclosure” refers any of the compounds described in Method 1 or the compounds described in any of the embodiments of Methods 1.1 to 1.71.

Fentanyl and fentanyl analogs are collectively referred to herein as “F/FA.” Fentanyl analogs include all chemical compounds recognized as-such by the U.S. Drug Enforcement Administration and/or by the United Nations Office on Drug and Crime (UNODC). F/FA compounds include, but are not limited to: fentanyl, alpha-methylfentanyl, 3-methylfentanyl, acetylfentanyl (also known as desmethylfentanyl), acetyl-alpha-methylfentanyl, thiofentanyl, alpha-methylthiofentanyl, beta-hydroxyfentanyl, para-fluorofentanyl, beta-hydroxy-3-methylthiofentanyl, beta-hydroxythiofentanyl, butyrylfentanyl, furanylfentanyl, 4-fluoroisobutyrylfentanyl, 4-fluorobutyrylfentanyl, 4-methoxybutyrylfentanyl, 4-methylbutyrylfentanyl, acrylfentanyl, 4-chloroisobutyrylfentanyl, tetrahydrofuranylfentanyl, cyclopentylfentanyl, valerylfentanyl, methoxyacetylfentanyl, 3-carbomethoxyfentanyl, sufentanil, alfentanil, remifentanil, carfentanil, thiafentanil, lofentanil, ocfentanil, trefantinil, brifentanil, AH-7921, U-47700, MT-45, and any other compounds “substantially similar” to fentanyl, sufentanil, alfentanil, remifentanil, or carfentanil. F/FA also include all drug compositions or mixtures containing any F/FA compound, such as morphine, heroin, codeine, hydrocodone, oxycodone, hydromorphone, marijuana or cannabis products, tetrahydrocannabinol, cocaine, amphetamine, methamphetamine, methylenedioxymethamphetamine, alprazolam, or other illicit or licit drugs, contaminated with or mixed with any F/FA compound described herein.

In further embodiments of the first aspect, the present disclosure provides further embodiments of Method 1 as follows:
1.85 Method 1 or any of Methods 1.1-1.84, wherein the patient is unconscious;
1.86 Method 1 or any of Methods 1.1-1.84, wherein the patient is suspected of suffering from an acute F/FA overdose;
1.87 Method 1 or any of Methods 1.1-1.86, wherein the patient demonstrates chest wall rigidity;
1.88 Method 1 or any of Methods 1.1-1.87, wherein the patient demonstrates laryngospasm;
1.89 Method 1 or any of Methods 1.1-1.88, wherein the patient is diagnosed with or suspected or having wooden chest syndrome (WCS);
1.90 Method 1 or any of Methods 1.1-1.88, wherein the patient is diagnosed with or suspected or having fentanyl-induced muscle rigidity (FIMR) or fentanyl-induced respiratory muscle rigidity (FIRMR) (wherein said FIMR or FIRMR is caused by fentanyl or by a fentanyl analog);
1.91 Method 1 or any of Methods 1.1-1.90, wherein the patient is in a non-hospital or non-emergency clinic setting;
1.92 Method 1 or any of Methods 1.1-1.91, wherein the patient is suspected of suffering from opioid use disorder, or has a history of opioid use disorder;
1.93 Method 1 or any of Methods 1.1-1.92, wherein the patient is suspected of being a naïve opioid user;
1.94 Method 1 or any of Methods 1.1-1.93, wherein the patient has, or is suspected of having, overdosed on a licit or illicit drug contaminated with or admixed with F/FA (e.g., morphine, heroin, codeine, hydrocodone, oxycodone, hydromorphone, marijuana or cannabis products, tetrahydrocannabinol, cocaine, amphetamine, methamphetamine, methylenedioxymethamphetamine, alprazolam, or other illicit or licit drugs);
1.95 Method 1, or any of Methods 1.1-1.94, wherein the F/FA is or was administered as general anesthesia (e.g., surgical anesthesia);
1.96 Method 1.95, wherein the general anesthesia further comprises or comprised one or more of an inhalational anesthetic (e.g., isoflurane, sevoflurane, desflurane, nitrous oxide, halothane, methoxyflurane), another opioid agonist (e.g., morphine, oxycodone), a sedative or hypnotic (e.g., propofol, midazolam, ketamine, etomidate), or a muscle relaxant (e.g., atracurium, mivacurium, pancuronium, rocuronium, vecuronium, cistracurium, succinylcholine);
1.97 Method 1.95 or 1.96, wherein the patient has difficulty emerging from anesthesia, e.g., due to persistent respiratory depression;
1.98 Method 1 or any of Methods 1.1-1.97, wherein the patient has not responded to, or has not responded adequately to (e.g., with respect to signs or symptoms of respiratory depression) a single dose of a mu-opioid antagonist (e.g., naloxone or naltrexone, e.g., 0.1 to 4 mg) administered by any route (e.g., intranasal, intravenous, subcutaneous, or intramuscular);
1.99 Method 1 or any of Methods 1.1-1.97, wherein the patient has not responded to, or has not responded adequately to (e.g., with respect to signs or symptoms of respiratory depression) multiple doses of a mu-opioid antagonist (e.g., naloxone or naltrexone, e.g., 0.4 to 20 mg in total) administered by any route (e.g., intranasal, intravenous, subcutaneous, or intramuscular);
1.100 Method 1 or any of Methods 1.1-1.99, wherein the patient has experienced recrudescence of respiratory depression following a single dose or multiple doses of a mu-opioid antagonist administered by any route (e.g., naloxone or naltrexone, e.g., intranasal, intravenous, subcutaneous, or intramuscular);
1.101 Method 1 or any of Methods 1.1-1.100, wherein the patient has experienced one or more opioid withdrawal symptoms or other adverse events (e.g., agitation, combativeness, laryngospasm, pulmonary edema, hemodynamic instability, or cardiac arrythmia) following a single dose or multiple doses of a mu-opioid antagonist administered by any route (e.g., naloxone or naltrexone, e.g., intranasal, intravenous, subcutaneous, or intramuscular);
1.102 Any of Methods 1.98-1.101, wherein said mu-opioid antagonist is naloxone, naltrexone, or nalmefene;
1.103 Any of Methods 1.98-1.101, wherein said mu-opioid antagonist is naloxone;
1.104 Method 1 or any of 1.1-1.103, wherein the patient has been administered at least one dose of naloxone and has suffered from one or more opioid withdrawal symptoms or adverse events such that further doses of naloxone are contraindicated;
1.105 Method 1 or any of 1.1-1.104, wherein the use of naloxone is contraindicated for any reason;
1.106 Method 1 or any of 1.1-1.105, wherein said patient has previously suffered from an opioid overdose;
1.107 Method 1 or any of 1.1-1.106, wherein said patient is confirmed to be suffering from F/FA overdose by toxicological or forensic methods (e.g., by confirming the presence of F/FA in the patient's blood, or in the patient's drugs or drug paraphernalia);
1.108 Method 1 or any of 1.1-1.107, wherein the effective amount of the Compound of Formula I is an amount effective to reverse one or more of: respiratory arrest, respiratory depression, skeletal muscle spasm, chest wall rigidity, laryngospasm, pupillary constriction, cardiac arrest, bradycardia, or unconsciousness;
1.109 Method 1 or any of 1.1-1.108, wherein the effective amount of the Compound of Formula I is 0.1 mg-200 mg, for example, 1-200 mg, or 10-150 mg, or 25-100 mg, or 50-100 mg, or 75-100 mg, or 25-75 mg, or 25-50 mg, or 1-50 mg, or 1-25 mg, 0.1 to 50 mg, 2.5 mg-50 mg, or for a long-acting formulation, 25 mg-1500 mg, for example, 50 mg to 500 mg, or 250 mg to 1000 mg, or 250 mg to 750 mg, or 75 mg to 300 mg;
1.110 Method 1.109, wherein the effective amount is administered as a single dose;
1.111 Method 1.109, wherein the effective amount is administered in two or more doses over a period of less than 30 minutes (e.g., less than 20 minutes, or less than 15 minutes, or less than 10 minutes);
1.112 Method 1 or any of 1.1-1.111, wherein the effective amount of the Compound of Formula I is administered by intranasal administration (e.g., as an aerosol, mist, or powder for inhalation);
1.113 Method 1 or any of 1.1-1.111, wherein the effective amount of the Compound of Formula I is administered across the oral mucosa, such as, by a rapidly-dissolving oral tablet (e.g., a rapidly dissolving sublingual tablet);
1.114 Method 1 or any of 1.1-1.111, wherein the effective amount of the Compound of Formula I is administered by injection (e.g., intravenous, intramuscular, intrathecal, intraperitoneal, or subcutaneous injection);
1.115 Any foregoing method, wherein the method does not comprise the concurrent administration of any other opioid antagonist (e.g., naloxone, naltrexone, nalmefene, methadone, nalorphine, levallorphan, samidorphan, nalodeine, cyprodime, or norbinaltorphimine);
1.116 Any foregoing method wherein the method comprises administering a pharmaceutical composition comprising both a Compound of Formula I, wherein R¹ is H, and a prodrug of the same Compound of Formula I (i.e., wherein R¹ is —C(O)—O—C(R^{a})(R^{b})(R^{c}), —C(O)—O—CH₂—O—C(R^{a})(R^{b})(R^{c}) or —C(R⁶)(R⁷)—O—C(O)—R^{g}, as hereinbefore described);
1.117 Any foregoing method wherein the Compound of Formula I is the sole pharmacological treatment for the overdose (e.g., other than supportive interventions, such as oxygen administration, cardiopulmonary resuscitation, chest compressions, and fluid administration);
1.118 Any foregoing method, wherein the method is a method for treating or reversing F/FA overdose;
1.119 Any foregoing method, wherein the method is a method for treating or reversing F/FA-induced respiratory depression;
1.120 Any foregoing method, wherein the method is a method for treating or reversing F/FA-induced muscle rigidity;
1.121 Any foregoing method, wherein the method is a method for treating or reversing F/A-induced laryngospasm;
1.122 Any foregoing method, wherein the method is a method for reversing or inhibiting binding of F/FA to mu-opioid receptors in the central nervous system (e.g., in the locus coeruleus);
1.123 Any foregoing method, wherein the method is a method for inhibiting F/FA-induced beta-arrestin signaling in the central nervous system (e.g., in the locus coeruleus);
1.124 Any foregoing method, wherein the method is a method for preventing death from F/FA overdose;
1.125 Any foregoing method, wherein the method is a method for anesthetic recovery (e.g., anesthetic emergence, such as, following surgery) 1.126 Any foregoing method, wherein the F/FA is any F/FA disclosed herein throughout;
1.127 Any foregoing method, wherein the F/FA is selected from fentanyl, sufentanil, alfentanil, remifentanil, carfentanil, thiafentanil, lofentanil, ocfentanil, trefantinil, and brifentanil;
1.128 Any foregoing method, wherein the F/FA is selected from fentanyl, sufentanil, alfentanil and carfentanil;
1.129 Any foregoing method, wherein the F/FA is fentanyl;
1.130 Any foregoing method, wherein the method does not cause precipitated withdrawal in the patient, e.g., withdrawal symptoms selected from tachycardia, nausea, vomiting, diarrhea, extreme anxiety, restless legs, muscle aches, and profuse sweating;
1.131 Any of Method 1, or 1.1-1.130, wherein the source of the F/FA is another illicit drug which is adulterated with the F/FA, such as cocaine, heroin, oxycodone, amphetamine, methamphetamine, or marijuana;
1.132 Any of Method 1 or 1.1-1.130, wherein the method is a method of:
   (a) treating or reversing a drug overdose;
   (b) treating or reversing drug-induced respiratory depression;
   (c) treating or reversing drug-induced muscle rigidity;
   (d) treating or reversing drug-induced laryngospasm; or
   (e) preventing death from drug overdose;
      wherein the drug is an illicit drug which has been adulterated with F/FA;
1.133 Method 1.132, wherein the illicit drug is heroin, cocaine, amphetamine, methamphetamine, oxycodone, or marijuana;
1.134 Method 1.132 or 1.133, wherein the overdose, respiratory depression, muscle rigidity, laryngospasm, and/or risk of death is due primarily or exclusively to the F/FA adulterant in the illicit drug (e.g., but for the F/FA adulterant, said illicit drug would not have caused said overdose, respiratory depression, muscle rigidity, laryngospasm, and/or risk of death).

In any of the embodiments of Method 1 et seq. wherein the Compound of the present disclosure is administered along with one or more second therapeutic agents, the one or more second therapeutic agents may be administered as a part of the pharmaceutical composition comprising the Compound of the present disclosure. Alternatively, the one or more second therapeutic agents may be administered in separate pharmaceutical compositions (such as pills, tablets, capsules and injections) administered simultaneously, sequentially or separately from the administration of the Compound of the present disclosure.

In a second aspect, the present disclosure provides use of a Compound of the present disclosure, e.g., a Compound of Formula I or any of the compounds described in any of the embodiments of Methods 1.1 to 1.71, in the manufacture of a medicament for use according to Method 1 or any of Methods 1.1-1.134.

In a third aspect, the present disclosure provides a Compound of the present disclosure, e.g., a Compound of Formula I or any of the compounds described in any of the embodiments of Methods 1.1 to 1.71, for use according to Method 1 or any of Methods 1.1-1.134.

Without being bound by theory, it is believed that the Compounds of the present disclosure, such as the Compound of Formula A, due to their potent 5-HT_{2A}, D₁ and Mu opioid modulation activity, and especially due to their biased mu-opioid receptor activity, are unexpectedly effective in reversing the symptoms of F/FA overdose, especially respiratory depression, chest wall rigidity and laryngospasm. This is particularly believed to be due to these compounds' activity as mu-receptor antagonists via the beta-arrestin signaling. It is also believed that these compounds' activity as alpha1-adrenergic antagonists, as indirect NMDA and AMPA antagonists, and potentially due to indirect effects on GABA expressing neurons. These properties are highly unique and are not shared by the traditional mu-opioid receptor antagonists which are used for both opioid overdose treatment and surgical reversal of opioid agonism, such as naloxone.

The compounds disclosed herein are also highly beneficial in treating acute overdose and chronic opioid addiction because they do not induce opioid withdrawal symptoms in the manner that opioid cessation or opioid antagonist treatment may. Opioid withdrawal syndrome can be very severe on addicted patients, and can include symptoms such as tachycardia, nausea, vomiting, diarrhea, extreme anxiety, restless legs, muscle aches, and profuse sweating. These withdrawal symptoms are the result of the body's adaptation to the presence of opioids resulting in tolerance and physical dependence. In severe cases, sudden cessation of opioid abuse or treatment with opioid antagonists can result in withdrawal symptoms lasting for weeks or months. Administration of opioid antagonists, such as naloxone or naltrexone, especially in high doses, can precipitate acute withdrawal effects, especially in patients suffering from an acute overdose with F/FA. In patients suffering from overdose with weaker opioid agonists, such as heroin, antagonist treatment can be administered using small repeated doses in order to avoid or minimize such withdrawal syndromes. However, in an acute F/FA overdose, such small doses of antagonist are ineffective, and thus, in order to have any chance of reversing the overdose, it is often impossible to avoid severe withdrawal with traditional antagonist treatments.

In some embodiments of the present disclosure, the compounds of Formula I have one or more biologically labile functional groups positioned within the compounds such that natural metabolic activity will remove the labile functional groups, resulting in another Compound of Formula I. For example, when group R¹ is C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, under biological conditions this substituent will undergo hydrolysis to yield the same compound wherein R¹ is H, thus making the original compounds prodrugs of the compound wherein R¹ is H. Some of such prodrug compounds may have little-to-no or only moderate biological activity but upon hydrolysis to the compound wherein R¹ is H, the compound may have strong biological activity. As such, depending on the compound selected, administration of the compounds of the present disclosure to a patient in need thereof may result in immediate biological and therapeutic effect, or immediate and delayed biological and therapeutic effect, or only delayed biological and therapeutic effect. Such prodrug compounds will thus serve as a reservoir of the pharmacologically active compounds of Formula I wherein R¹ is H. In particular embodiments, the nature of the group R¹ may be such that the resulting compound of Formula I is substantially more lipophilic than the corresponding compound of Formula I wherein R¹ is H, and as a result, the prodrug compound may cross the blood brain barrier and accumulate in the central nervous system (CNS) tissues much more rapidly, followed by rapid hydrolysis of the labile group win the CNS. Overall, this may result in more rapid action of the compound to reverse the effects of mu-receptor activation.

In another embodiment, the methods of the present disclosure provide for the administration of a pharmaceutical composition comprising both a Compound of Formula I and a prodrug of the same compound. Thus, the composition may comprise a particular compound of Formula I wherein R¹ is H, and a second compound of Formula 1 wherein R¹ is —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸, but wherein the compounds are otherwise the same. In such an embodiment, depending on the nature of the group R¹ of the prodrug, the composition may provide an immediate release effect owing to rapid absorption and action of the compound of Formula I wherein R¹ is H, combined with a sustained or delayed release effect owing to the slower hydrolysis of the prodrug version of the compound, generating additional compound of Formula I wherein R¹ is H over a course of time (e.g., 1-3 hours, 6-12 hours, 12-48 hours, 2-3 days). Therefore, in a particular aspect, the present disclosure also provides a pharmaceutical composition comprising a first compound of Formula I, wherein R¹ is H, and a second compound of Formula 1, wherein R¹ is —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸. In further embodiments of this aspect, both the compound of Formula I wherein R¹ is H, and the prodrug compound of Formula I may be as described in any of embodiments 1.1-1.70, and the pharmaceutical composition comprising same may be as described in any other pharmaceutical composition embodiments described herein.

“Alkyl” as used herein is a saturated or unsaturated hydrocarbon moiety, e.g., one to twenty-one carbon atoms in length, unless indicated otherwise; any such alkyl may be linear or branched (e.g., n-butyl or tert-butyl), preferably linear, unless otherwise specified. For example, “C₁₋₂₁ alkyl” denotes alkyl having 1 to 21 carbon atoms. In one embodiment, alkyl is optionally substituted with one or more hydroxy or C₁₋₂₂alkoxy (e.g., ethoxy) groups. In another embodiment, alkyl contains 1 to 21 carbon atoms, preferably straight chain and optionally saturated or unsaturated, for example in some embodiments wherein R₁ is an alkyl chain containing 1 to 21 carbon atoms, preferably 6-15 carbon atoms, 16-21 carbon atoms, e.g., so that together with the —C(O)— to which it attaches, e.g., when cleaved from the compound of Formula I, forms the residue of a natural or unnatural, saturated or unsaturated fatty acid.

The term “pharmaceutically acceptable diluent or carrier” is intended to mean diluents and carriers that are useful in pharmaceutical preparations, and that are free of substances that are allergenic, pyrogenic or pathogenic, and that are known to potentially cause or promote illness. Pharmaceutically acceptable diluents or carriers thus exclude bodily fluids such as example blood, urine, spinal fluid, saliva, and the like, as well as their constituent components such as blood cells and circulating proteins. Suitable pharmaceutically acceptable diluents and carriers can be found in any of several well-known treatises on pharmaceutical formulations, for example Goodman and Gilman, eds., The Pharmacological Basis of Therapeutics, Tenth Edition, McGraw Hill, 2001; Remington's Pharmaceutical Sciences, 20th Ed., Lippincott Williams & Wilkins., 2000; and Martindale, The Extra Pharmacopoeia, Thirty-Second Edition (The Pharmaceutical Press, London, 1999); all of which are incorporated by reference herein in their entirety.

The terms “purified,” “in purified form” or “in isolated and purified form” for a compound refers to the physical state of said compound after being isolated from a synthetic process (e.g., from a reaction mixture), or natural source or combination thereof. Thus, the term “purified,” “in purified form” or “in isolated and purified form” for a compound refers to the physical state of said compound after being obtained from a purification process or processes described herein or well known to the skilled artisan (e.g., chromatography, recrystallization, LC-MS and LC-MS/MS techniques and the like), in sufficient purity to be characterizable by standard analytical techniques described herein or well known to the skilled artisan.

Unless otherwise indicated, the Compounds of the present disclosure may exist in free base form or in salt form, such as a pharmaceutically acceptable salt form, e.g., as acid addition salts. An acid-addition salt of a compound of the present disclosure which is sufficiently basic, for example, an acid-addition salt with, for example, an inorganic or organic acid, for example hydrochloric acid or toluenesulfonic acid. In addition, a salt of a compound of the present disclosure which is sufficiently acidic is an alkali metal salt, for example a sodium or potassium salt, or a salt with an organic base which affords a physiologically-acceptable cation. In a particular embodiment, the salt of the Compounds of the present disclosure is a toluenesulfonic acid addition salt.

The Compounds of the present disclosure are intended for use as pharmaceuticals, therefore pharmaceutically acceptable salts are preferred. Salts which are unsuitable for pharmaceutical uses may be useful, for example, for the isolation or purification of free Compounds of the present disclosure, and are therefore also included within the scope of the compounds of the present disclosure.

The Compounds of the present disclosure may comprise one or more chiral carbon atoms. The compounds thus exist in individual isomeric, e.g., enantiomeric or diastereomeric form or as mixtures of individual forms, e.g., racemic/diastereomeric mixtures. Any isomer may be present in which the asymmetric center is in the (R)-, (S)-, or (R,S)-configuration. The invention is to be understood as embracing both individual optically active isomers as well as mixtures (e.g., racemic/diastereomeric mixtures) thereof. Accordingly, the Compounds of the present disclosure may be a racemic mixture or it may be predominantly, e.g., in pure, or substantially pure, isomeric form, e.g., greater than 70% enantiomeric/diastereomeric excess (“ee”), preferably greater than 80% ee, more preferably greater than 90% ee, most preferably greater than 95% ee. The purification of said isomers and the separation of said isomeric mixtures may be accomplished by standard techniques known in the art (e.g., column chromatography, preparative TLC, preparative HPLC, simulated moving bed and the like).

Geometric isomers by nature of substituents about a double bond or a ring may be present in cis (Z) or trans (E) form, and both isomeric forms are encompassed within the scope of this invention.

It is also intended that the compounds of the present disclosure encompass their stable and unstable isotopes. Stable isotopes are nonradioactive isotopes which contain one additional neutron compared to the abundant nuclides of the same species (i.e., element). It is expected that the activity of compounds comprising such isotopes would be retained, and such compound would also have utility for measuring pharmacokinetics of the non-isotopic analogs. For example, the hydrogen atom at a certain position on the compounds of the disclosure may be replaced with deuterium (a stable isotope which is non-radioactive). Examples of known stable isotopes include, but not limited to, deuterium (2H or D), ¹³C, ¹⁵N, 180. Alternatively, unstable isotopes, which are radioactive isotopes which contain additional neutrons compared to the abundant nuclides of the same species (i.e., element), e.g., ¹²³I, ¹³¹I, ¹²⁵I, ¹¹C, ¹⁸F, may replace the corresponding abundant species of I, C and F. Another example of useful isotope of the compound of the present disclosure is the ¹¹C isotope. These radio isotopes are useful for radio-imaging and/or pharmacokinetic studies of the compounds of the present disclosure. In addition, the substitution of atoms of having the natural isotopic distributing with heavier isotopes can result in desirable change in pharmacokinetic rates when these substitutions are made at metabolically liable sites. For example, the incorporation of deuterium (²H) in place of hydrogen can slow metabolic degradation when the position of the hydrogen is a site of enzymatic or metabolic activity.

An “effective amount” means a “therapeutically effective amount”, that is, any amount of the Compounds of the present disclosure (for example as contained in the pharmaceutical composition or dosage form) which, when administered to a subject suffering from a disease or disorder, is effective to cause a reduction, remission, or regression of the disease or disorder over the period of time as intended for the treatment.

Dosages employed in practicing the present invention will of course vary depending, e.g., on the particular disease or condition to be treated, the particular Compound of the present disclosure used, the mode of administration, and the therapy desired. Unless otherwise indicated, an amount of the Compound of the present disclosure for administration (whether administered as a free base or as a salt form) refers to or is based on the amount of the Compound of the present disclosure in free base form (i.e., the calculation of the amount is based on the free base amount).

Compounds of the present disclosure may be administered by any satisfactory route, including orally, parenterally (intravenously, intramuscular or subcutaneous) or transdermally. In certain embodiments, the Compounds of the present disclosure, e.g., in depot formulation, is preferably administered parenterally, e.g., by injection, for example, intramuscular or subcutaneous injection.

The pharmaceutically acceptable salts of the Compounds of the present disclosure can be synthesized from the parent compound which contains a basic or acidic moiety by conventional chemical methods. Generally, such salts can be prepared by reacting the free base forms of these compounds with a stoichiometric amount of the appropriate acid in water or in an organic solvent, or in a mixture of the two; generally, non-aqueous media like ether, ethyl acetate, ethanol, isopropanol, or acetonitrile are preferred.

Pharmaceutical compositions comprising Compounds of the present disclosure may be prepared using conventional diluents or excipients (an example include, but is not limited to sesame oil) and techniques known in the galenic art. Thus, oral dosage forms may include tablets, capsules, solutions, suspensions and the like.

The term “concurrently” when referring to a therapeutic use means administration of two or more active ingredients to a patient as part of a regimen for the treatment of a disease or disorder, whether the two or more active agents are given at the same or different times or whether given by the same or different routes of administrations. Concurrent administration of the two or more active ingredients may be at different times on the same day, or on different dates or at different frequencies.

The term “simultaneously” when referring to a therapeutic use means administration of two or more active ingredients at or about the same time by the same route of administration.

The term “separately” when referring to a therapeutic use means administration of two or more active ingredients at or about the same time by different route of administration.

### Methods of Making the Compounds of the Present Disclosure:

The Compound of Formula A, and methods for its synthesis, including the synthesis of intermediates used in the synthetic schemes described below, have been disclosed in, for example, U.S. Pat. Nos. 8,309,722, and 10,245,260, US 2021/00009592, and WO2020/131895. The synthesis of similar fused gamma-carbolines has been disclosed in, for example, U.S. Pat. Nos. 8,309,722, 8,993,572, 10,077,267, 10,961,245, 10,906,906, US 2021/0163481, and WO 2020/132605 (US 2022/0048910), the contents of each of which are incorporated by reference in their entireties. Compounds of the present disclosure can be prepared using similar procedures.

Isolation or purification of the diastereomers of the Compounds of the present disclosure may be achieved by conventional methods known in the art, e.g., column purification, preparative thin layer chromatography, preparative HPLC, crystallization, trituration, chiral salt resolution, simulated moving beds and the like.

Salts of the Compounds of the present disclosure may be prepared as similarly described in U.S. Pat. Nos. 6,552,017; 7,183,282; 8,648,077; 10,654,854; and 11,014,925; the contents of each of which are incorporated by reference in their entirety.

Diastereomers of prepared compounds can be separated by, for example, HPLC. For example, a CHIRALPAK® AY-H, 5μ, 30×250 mm column operated at room temperature and eluted with an ethanol/hexane/dimethylethylamine solvent system can be used.

### EXAMPLES

### Example 1: Synthesis of (6bR,10aS)-8-(3-(4-fluorophenoxy)propyl)-6b,7,8,9,10,10a-hexahydro-1H-pyrido[3′,4′:4,5]pyrrolo[1,2,3-de]quinoxalin-2(3H)-one

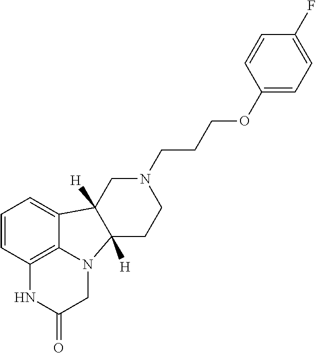 [SMILES: O=C1CN2c3c(cccc3[C@@H]3CN(CCCOc4ccc(F)cc4)CC[C@@H]32)N1]

A mixture of (6bR,10aS)-6b,7,8,9,10,10a-hexahydro-1H-pyrido[3′,4′:4,5]pyrrolo[1,2,3-de]quinoxalin-2(3H)-one (100 mg, 0.436 mmol), 1-(3-chloroproxy)-4-fluorobenzene (100 μL, 0.65 mmol) and potassium iodide (KI) (144 mg, 0.87 mmol) in dimethylformamide (DMF) (2 mL) is degassed with argon for 3 minutes and N,N-diisopropylethylamine (DIPEA) (150 μL, 0.87 mmol) is added. The resulting mixture is heated to 78° C. and stirred at this temperature for 2 h. The mixture is cooled to room temperature and then filtered. The filter cake is purified by silica gel column chromatography using a gradient of 0-100% ethyl acetate in a mixture of methanol/7N NH₃ in methanol (1:0.1 v/v) as an eluent to produce partially purified product, which is further purified with a semi-preparative HPLC system using a gradient of 0-60% acetonitrile in water containing 0.1% formic acid over 16 min to obtain the title product as a solid (50 mg, yield 30%). MS (ESI) m/z 406.2 [M+1]+. ¹H NMR (500 MHz, DMSO-d₆) δ 10.3 (s, 1H), 7.2-7.1 (m, 2H), 7.0-6.9 (m, 2H), 6.8 (dd, J=1.03, 7.25 Hz, 1H), 6.6 (t, J=7.55 Hz, 1H), 6.6 (dd, J=1.07, 7.79 Hz, 1H), 4.0 (t, J=6.35 Hz, 2H), 3.8 (d, J=14.74 Hz, 1H), 3.3-3.2 (m, 3H), 2.9 (dd, J=6.35, 11.13 Hz, 1H), 2.7-2.6 (m, 1H), 2.5-2.3 (m, 2H), 2.1 (t, J=11.66 Hz, 1H), 2.0 (d, J=14.50 Hz, 1H), 1.9-1.8 (m, 3H), 1.7 (t, J=11.04 Hz, 1H).

### Example 2: Synthesis of (6bR,10aS)-8-(3-(6-fluoro-1H-indazol-3-yl)propyl)-6b,7,8,9,10,10a-hexahydro-1H-pyrido[3′,4′:4,5]pyrrolo[1,2,3-de]quinoxalin-2(3H)-one

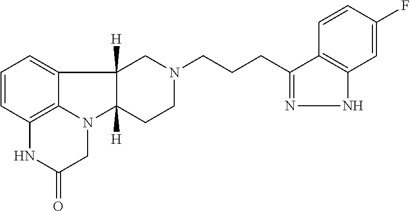 [SMILES: O=C1CN2c3c(cccc3[C@@H]3CN(CCCc4n[nH]c5cc(F)ccc45)CC[C@@H]32)N1]

Step 1: To a stirred solution of BCl₃·MeS (10.8 g, 60 mmol) in toluene at 0-5° C. is added 3-fluoroaniline (5.6 mL, 58 mmol), followed by 4-chlorobutyronitrile (7.12 g. 68.73 mmol) and aluminum chloride (AlCl₃) (8.0 g, 60.01 mmol). The mixture is stirred at 130° C. overnight and cooled to 50° C. Hydrochloric acid (3N, 30 mL) is added carefully and the resulting solution is stirred at 90° C. overnight. The obtained brown solution is cooled to room temperature and evaporated to dryness. The residue is dissolved in dichloromethane (DCM) (20 mL) and basified with saturated Na₂CO₃ to pH=7-8. The organic phase is separated, dried over Na₂CO₃ and then concentrated. The residue is purified by silica-gel column chromatography using a gradient of 0-20% ethyl acetate in hexane as eluent to afford 2′-amino-4-chloro-4′-fluorobutyrophenone as a yellow solid (3.5 g, yield 28%). MS (ESI) m/z 216.1 [M+1]⁺.
Step 2: To a suspension of 2′-amino-4-chloro-4′-fluorobutyrophenone (680 mg, 3.2 mmol) in concentrated HCl (14 mL) at 0-5° C., NaNO₂ (248 mg, 3.5 mmol) in water (3 mL) is added. The resulting brown solution is stirred at 0-5° C. for 1 h and then SnCl₂·2H₂O (1.74 g, 7.7 mmol) in concentrated HCl (3 mL) is added. The mixture is stirred at 0-5° C. for additional 1 hour and then dichloromethane (30 mL) is added. The reaction mixture is filtered and the filtrate is dried over K₂CO₃ and evaporated to dryness. The residue is purified by silica-gel column chromatography using a gradient of 0-35% ethyl acetate in hexane as eluent to yield 3-(3-chloropropyl)-6-fluoro-1H-indazole as a white solid (400 mg, yield 60%). MS (ESI) m/z 213.1 [M+1]⁺.
Step 3: A mixture of (6bR,10aS)-6b,7,8,9,10,10a-hexahydro-1H-pyrido[3′,4′:4,5]pyrrolo[1,2,3-de]quinoxalin-2(3H)-one (100 mg, 0.436 mmol), 3-(3-chloropropyl)-6-fluoro-1H-indazole(124 mg, 0.65 mmol) and KI (144 mg, 0.87 mmol) is degassed with argon for 3 minutes and DIPEA (150 μL, 0.87 mmol) is added. The resulting mixture is stirred at 78° C. for 2 h and then cooled to room temperature. The generated precipitate is filtered. The filter cake is purified with a semi-preparative HPLC system using a gradient of 0-60% acetonitrile in water containing 0.1% formic acid over 16 minutes to yield (6bR,10aS)-8-(3-(6-fluoro-1H-indazol-3-yl)propyl)-6b,7,8,9,10,10a-hexahydro-1H-pyrido[3′,4′:4,5]pyrrolo[1,2,3-de]quinoxalin-2(3H)-one as an off-white solid (50 mg, yield 28%). MS (ESI) m/z 406.2 [M+1]⁺. ¹H NMR (500 MHz, DMSO-d₆) δ 12.7 (s, 1H), 10.3 (s, 1H), 7.8 (dd, J=5.24, 8.76 Hz, 1H), 7.2 (dd, J=2.19, 9.75 Hz, 1H), 6.9 (ddd, J=2.22, 8.69, 9.41 Hz, 1H), 6.8-6.7 (m, 1H), 6.6 (t, J=7.53 Hz, 1H), 6.6 (dd, J=1.07, 7.83 Hz, 1H), 3.8 (d, J=14.51 Hz, 1H), 3.3-3.2 (m, 1H), 3.2 (s, 2H), 2.9 (dt, J=6.35, 14.79 Hz, 3H), 2.7-2.6 (m, 1H), 2.4-2.2 (m, 2H), 2.1 (t, J=11.42 Hz, 1H), 2.0-1.8 (m, 3H), 1.8-1.7 (m, 1H), 1.7 (t, J=10.89 Hz, 1H).

### Example 3: Synthesis of (6bR,10aS)-8-(3-(6-fluorobenzo[d]isoxazol-3-yl)propyl)-6b,7,8,9,10,10a-hexahydro-1H-pyrido[3′,4′:4,5]pyrrolo[1,2,3-de]quinoxalin-2(3H)-one

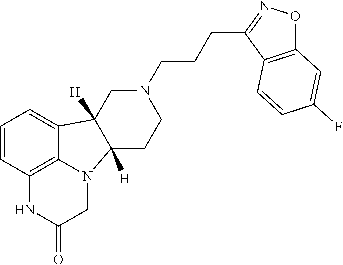 [SMILES: O=C1CN2c3c(cccc3[C@@H]3CN(CCCc4noc5cc(F)ccc45)CC[C@@H]32)N1]

A mixture of (6bR,10aS)-6b,7,8,9,10,10a-hexahydro-1H-pyrido[3′,4′:4,5]pyrrolo[1,2,3-de]quinoxalin-2(3H)-one (148 mg, 0.65 mmol), 3-(3-chloropropyl)-6-fluorobenzo[d]isoxazole (276 mg, 1.3 mmol) and KI (210 mg, 1.3 mmol) is degassed with argon and then DIPEA (220 μL, 1.3 mmol) is added. The resulting mixture is stirred at 78° C. for 2 h and then cooled to room temperature. The mixture is concentrated under vacuum. The residue is suspended in dichloromethane (50 mL) and then washed with water (20 mL). The organic phase is dried over K₂CO₃, filtered, and then concentrated under vacuum. The crude product is purified by silica gel column chromatography with a gradient of 0-10% of methanol in ethyl acetate containing 1% 7N NH₃ to yield the title product as a solid (80 mg, yield 30%). MS (ESI) m/z 407.2 [M+1]⁺. ¹H NMR (500 MHz, DMSO-d₆) δ 10.3 (s, 1H), 8.0-7.9 (m, 1H), 7.7 (dd, J=2.15, 9.19 Hz, 1H), 7.3 (td, J=2.20, 9.09 Hz, 1H), 6.8 (d, J=7.22 Hz, 1H), 6.6 (t, J=7.54 Hz, 1H), 6.6 (d, J=7.75 Hz, 1H), 3.8 (d, J=14.53 Hz, 1H), 3.3 (s, 1H), 3.2 (s, 1H), 3.2-3.1 (m, 1H), 3.0 (t, J=7.45 Hz, 2H), 2.9-2.8 (m, 1H), 2.7-2.5 (m, 1H), 2.4-2.2 (m, 2H), 2.2-2.0 (m, 1H), 2.0-1.8 (m, 3H), 1.8-1.6 (m, 2H).

### Example 4: Synthesis of 4-(3-((6bR,10aS)-2-oxo-2,3,6b,7,10,10a-hexahydro-1H-pyrido[3′,4′:4,5]-pyrrolo[1,2,3-de]quinoxalin-8(9H)-yl)propoxy)benzonitrile

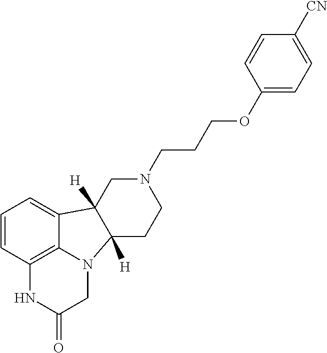 [SMILES: N#Cc1ccc(OCCCN2CC[C@H]3[C@@H](C2)c2cccc4c2N3CC(=O)N4)cc1]

Step 1: A degassed suspension of (4aS,9bR)-ethyl 6-bromo-3,4,4a,5-tetrahydro-1H-pyrido[4,3-b]indole-2(9bH)-carboxylate (21.5 g, 66.2 mmol), chloroacetamide (9.3 g, 100 mmol), and KI (17.7 g, 107 mmol) in dioxane (60 mL) is stirred at 104° C. for 48 h. The solvent is removed and the residue is suspended in dichloromethane (200 mL) and extracted with water (100 mL). The separated dichloromethane phase is dried over potassium carbonate (K₂CO₃) for 1 h and then filtered. The filtrate is evaporated to give a crude product as a brown oil. To the brown oil is added ethyl acetate (100 m L) and the mixture is sonicated for 2 min. A yellow solid gradually precipitates from the mixture, which turns into a gel after standing at room temperature for an additional 2 h. Additional ethyl acetate (10 mL) is added and the resulting solid is filtered. The filtered cake is rinsed with ethyl acetate (2 m L) and further dried under high vacuum to produce (4aS,9bR)-ethyl 5-(2-amino-2-oxoethyl)-6-bromo-3,4,4a,5-tetrahydro-1H-pyrido[4,3-b]indole-2(9bH)-carboxylate as an off white solid (19 g, yield 75%). This product is used directly in the next step without further purification. MS (ESI) m/z 382.0 [M+H]⁺.
Step 2: A mixture of (4aS,9bR)-ethyl 5-(2-amino-2-oxoethyl)-6-bromo-3,4,4a,5-tetrahydro-1H-pyrido[4,3-b]indole-2(9bH)-carboxylate (12.9 g, 33.7 mmol), KI (10.6 g, 63.8 mmol), CuI (1.34 g, 6.74 mmol) in dioxane (50 mL) is bubbled with argon for 5 min. To this mixture is added N,N,N,N′-tetramethylethylenediamine (3 mL) and the resulting suspension is stirred at 100° C. for 48 h. The reaction mixture is cooled to room temperature and poured onto a silica gel pad to filter. The filtered cake is rinsed with ethyl acetate (1L×2). The combined filtrate is concentrated to dryness to give a product (6bR,10aS)-2-oxo-2,3,6b,9,10,10a-hexahydro-1H,7H-pyrido[3′,4′:4,5]pyrrolo[1,2,3-de]quinoxaline-8-carboxylic acid ethyl esters a white solid (8 g, yield 79%). MS (ESI) m/z 302.1 [M+H]+.
Step 3: (6bR,10aS)-2-oxo-2,3,6b,9,10,10a-hexahydro-1H,7H-pyrido[3′,4′:4,5]pyrrolo[1,2,3-de]quinoxaline-8-carboxylic acid ethyl ester (6.4 g, 21.2 mmol) is suspended in HBr/acetic acid solution (64 mL, 33% w/w) at room temperature. The mixture is heated at 50° C. for 16 h. After cooling and treatment with ethyl acetate (300 mL), the mixture is filtered. The filter cake is washed with ethyl acetate (300 mL), and then dried under vacuum. The obtained HBr salt is then suspended in methanol (200 mL) and cooled with dry ice in isopropanol. Under vigorous stirring, ammonia solution (10 mL, 7N in methanol) is added slowly to the suspension to adjust the pH of the mixture to 10. The obtained mixture is dried under vacuum without further purification to give crude (6bR,10aS)-2-oxo-2,3,6b,9,10,10a-hexahydro-1H,7H-pyrido[3′,4′:4,5]pyrrolo[1,2,3-de]quinoxaline (8.0 g), which is used directly in the next step. MS (ESI) m/z 230.2 [M+H]⁺.
Step 4: A mixture of (6bR,10aS)-6b,7,8,9,10,10a-hexahydro-1H-pyrido[3′,4′:4,5]pyrrolo[1,2,3-de]quinoxalin-2(3H)-one (100 mg, 0.436 mmol), 4-(3-bromopropoxy)benzonitrile (99 mg, 0.40 mmol) and KI (97 mg, 0.44 mmol) in DMF (2 mL) is bubbled with argon for 3 minutes and diisopropylethylamine (DIPEA) (80 μL, 0.44 mmol) is added. The resulting mixture is heated to 76° C. and stirred at this temperature for 2 h. The solvent is removed, and the residue is purified by silica gel column chromatography using a gradient of 0-100% mixed solvents [ethyl acetate/methanol/7N NH₃ (10:1:0.1 v/v)] in ethyl acetate to obtain the title product as a white foam (35 mg, yield 45%). MS (ESI) m/z 389.1 [M+1]⁺. ¹H NMR (500 MHz, DMSO-d₆) δ 10.3 (s, 1H), 7.8 (d, J=8.80 Hz, 2H), 7.1 (d, J=8.79 Hz, 2H), 6.8 (d, J=7.39 Hz, 1H), 6.6 (t, J=7.55 Hz, 1H), 6.6 (d, J=6.78 Hz, 1H), 4.1 (t, J=6.36 Hz, 2H), 3.8 (d, J=14.53 Hz, 1H), 3.3-3.2 (m, 3H), 3.0-2.8 (m, 1H), 2.7-2.6 (m, 1H), 2.5-2.3 (m, 2H), 2.2-2.0 (m, 1H), 2.0-1.8 (m, 3H), 1.8-1.7 (m, 1H), 1.7 (t, J=11.00 Hz, 1H).

### Example 5: Synthesis of (6bR,10aS)-8-(3-(4-chlorophenoxy)propyl)-6b,7,8,9,10,10a-hexahydro-1H-pyrido[3′,4′:4,5]pyrrolo[1,2,3-de]quinoxalin-2(3H)-one

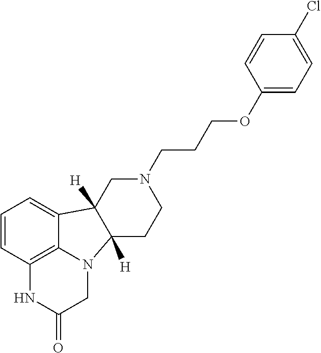 [SMILES: O=C1CN2c3c(cccc3[C@@H]3CN(CCCOc4ccc(Cl)cc4)CC[C@@H]32)N1]

To a degassed mixture of (6bR,10aS)-6b,7,8,9,10,10a-hexahydro-1H-pyrido[3′,4′:4,5]pyrrolo-[1,2,3-de]quinoxalin-2(3H)-one (110 mg, 0.48 mmol), 1-(3-bromopropoxy)-4-chlorobenzene (122 mg, 0.49 mmol) and KI (120 mg, 0.72 mmol) in DMF (2.5 mL) is added DIPEA (100 μL, 0.57 mmol). The resulting mixture is heated up to 76° C. and stirred at this temperature for 2 h. The solvent is removed, and the residue is purified by silica gel column chromatography using a gradient of 0-100% mixed solvents [ethyl acetate/methanol/7N NH₃ (10:1:0.1 v/v)] in ethyl acetate. The title product is given as a white solid (41 mg, yield 43%). (ESI) m/z 398.1 [M+1]⁺. ¹H NMR (500 MHz, DMSO-d₆) δ 10.3 (s, 1H), 7.4-7.2 (m, 2H), 6.9 (d, J=8.90 Hz, 2H), 6.8-6.7 (m, 1H), 6.6 (t, J=7.53 Hz, 1H), 6.6 (dd, J=1.04, 7.80 Hz, 1H), 4.0 (t, J=6.37 Hz, 2H), 3.8 (d, J=14.53 Hz, 1H), 3.3-3.2 (m, 3H), 2.9-2.8 (m, 1H), 2.7-2.6 (m, 1H), 2.4 (ddt, J=6.30, 12.61, 19.24 Hz, 2H), 2.1-2.0 (m, 1H), 2.0-1.9 (m, 1H), 1.9-1.7 (m, 3H), 1.7 (t, J=10.98 Hz, 1H).

### Example 6: Synthesis of (6bR,10aS)-8-(3-(quinolin-8-yloxy)propyl)-6b,7,8,9,10,10a-hexahydro-1H-pyrido[3′,4′:4,5]pyrrolo[1,2,3-de]quinoxalin-2(3H)-one

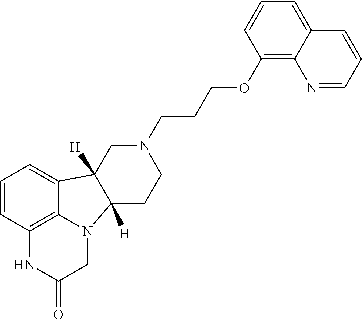 [SMILES: O=C1CN2c3c(cccc3[C@@H]3CN(CCCOc4cccc5cccnc45)CC[C@@H]32)N1]

A mixture of (6bR,10aS)-6b,7,8,9,10,10a-hexahydro-1H-pyrido[3′,4′:4,5]pyrrolo[1,2,3-de]quinoxalin-2(3H)-one (120 mg, 0.52 mmol), 8-(3-chloropropoxy)quinoline (110 mg, 0.50 mmol) and KI (120 mg, 0.72 mmol) in DMF (2.5 mL) is bubbled with argon for 3 minutes and DIPEA (100 μL, 0.57 mmol) is added. The resulting mixture is heated up to 76° C. and stirred at this temperature for 2 h. The solvent is removed, and the residue is suspended in dichloromethane (30 mL) and washed with water (10 mL). The dichloromethane phase is dried over K₂CO₃. The separated organic phase is evaporated to dryness. The residue is purified by silica gel column chromatography using a gradient of 0-100% mixed solvents [ethyl acetate/methanol/7N NH₃ (10:1:0.1 v/v)] in ethyl acetate to produce the title product as a light brown solid (56 mg, yield 55%). (ESI) m/z 415.2[M+1]*. ¹H NMR (500 MHz, DMSO-d₆) δ 10.1 (s, 1H), 8.9 (dd, J=1.68, 4.25 Hz, 1H), 8.3 (dd, J=1.71, 8.33 Hz, 1H), 7.7-7.5 (m, 3H), 7.3 (dd, J=1.50, 7.44 Hz, 1H), 7.0-6.8 (m, 1H), 6.8-6.5 (m, 2H), 4.4 (t, J=5.85 Hz, 2H), 3.9 (d, J=14.55 Hz, 1H), 3.8-3.6 (m, 2H), 3.5 (s, 1H), 3.4 (d, J=14.47 Hz, 1H), 2.9 (b, 1H), 2.3 (d, J=23.61 Hz, 5H), 1.3 (d, J=7.00 Hz, 3H).

### Example 7: Receptor Binding Profile

Receptor binding is determined for the Compound of Example 1 (the Compound of Formula A), and the Compounds of Examples 2 to 6. The following literature procedures are used, each of which reference is incorporated herein by reference in their entireties: 5-HT_{2A}: Bryant, H. U. et al. (1996), Life Sci., 15:1259-1268; D2: Hall, D. A. and Strange, P. G. (1997), Brit. J. Pharmacol., 121:731-736; D1: Zhou, Q. Y. et al. (1990), Nature, 347:76-80; SERT: Park, Y. M. et al. (1999), Anal. Biochem., 269:94-104; Mu opioid receptor: Wang, J. B. et al. (1994), FEBS Lett., 338:217-222.

In general, the results are expressed as a percent of control specific binding:

$$\frac{measured specific binding}{control specific binding} \times 100$$

and as a percent inhibition of control specific binding:

$$100 - ( \frac{measured specific binding}{control specific binding} \times 100 )$$

obtained in the presence of the test compounds.

The IC₅₀ values (concentration causing a half-maximal inhibition of control specific binding) and Hill coefficients (nH) are determined by non-linear regression analysis of the competition curves generated with mean replicate values using Hill equation curve fitting:

$$Y = D + [ \frac{A - D}{1 + {( C / {C}_{50} )}^{nH}} ]$$

where Y=specific binding, A=left asymptote of the curve, D=right asymptote of the curve, C=compound concentration, C₅₀=IC₅₀, and nH=slope factor. This analysis was performed using in-house software and validated by comparison with data generated by the commercial software SigmaPlot® 4.0 for Windows® (© 1997 by SPSS Inc.). The inhibition constants (Ki) were calculated using the Cheng Prusoff equation:

$$Ki = \frac{{IC}_{50}}{( 1 + L / {K}_{D} )}$$

where L=concentration of radioligand in the assay, and K_{D}=affinity of the radioligand for the receptor. A Scatchard plot is used to determine the K_{D}.

The following receptor affinity results are obtained:

| | Ki (nM) or maximum inhibition | | | | | |
|---|---|---|---|---|---|---|
| Receptor | Ex. 1 | Ex. 2 | Ex. 3 | Ex. 4 | Ex. 5 | Ex. 6 |
| 5-HT_{2A} | 8.3 | 2.6 | 3.1 | 0% @ | 15% @ | 0% @ |
| | | | | 10 nM | 10 nM | 10 nM |
| D2 | 160 | 15 | 84 | | | |
| D1 | 50 | 5.2 | 13 | 0% @ | 0% @ | 0% @ |
| | | | | 50 nM | 50 nM | 50 nM |
| SERT | 590 | 540 | | | | |
| Mu opioid receptor | 11 | 39 | 30 | 15 | 7.3 | 11 |

Additional compounds of Formula I are prepared by procedures analogous to those described in Examples 1-6. The receptor affinity results for these compounds are shown in the table below:

| | Compound Structure | | | | | | | | |
|---|---|---|---|---|---|---|---|---|---|
| L | —(CH₂)ₙX— | | | | | | | | |
| n | 4 | 2 | 3 | 3 | 3 | 3 | 3 | 3 | 3 |
| X | O | O | O | O | O | CH₂ | NH | N(CH₃) | S |
| Z | 4-F- | 4-F- | 4-MeO- | 4-F-3- | 4-F-2- | 4-F- | 4-F- | 4-F- | 4-F- |
| | phenyl | phenyl | phenyl | OH-phenyl | OH-phenyl | phenyl | phenyl | phenyl | phenyl |
| R¹ | H | H | H | H | H | H | H | H | H |
| R², R³ | H, H | H, H | H, H | H, H | H, H | H, H | H, H | H, H | H, H |

| Receptor | Ki (nM) or maximum inhibition | | | | | | | | |
|---|---|---|---|---|---|---|---|---|---|
| 5-HT_{2A} | 37% @ | 48% @ | 0% @ | 110 | 19 | 85% @ | 32% @ | 76% @ | 93% @ |
| | 100 nM | 100 nM | 100 nM | | | 100 nM | 100 nM | 100 nM | 100 nM |
| D2 | 27% @ | 24% @ | | 0% @ | 67 | 24% @ | 25% @ | 14% @ | 49% @ |
| | 100 nM | 100 nM | | 100 nM | | 100 nM | 100 nM | 100 nM | 100 nM |
| D1 | 5.4% @ | 10% @ | 0% @ | 25% @ | 22% @ | 32% @ | 11% @ | 21% @ | 54% @ |
| | 100 nM | 100 nM | 50 nM | 100 nM | 100 nM | 100 nM | 100 nM | 100 nM | 100 nM |
| SERT | 3.3% @ | 0% @ | 10% @ | 13% @ | 5% @ | 16% @ | 0% @ | 53% @ | 0% @ |
| | 100 nM | 100 nM | 100 nM | 100 nM | 100 nM | 200 nM | 200 nM | 200 nM | 200 nM |
| Mu | 39% @ | 30% @ | 0% @ | 23% @ | 22% @ | 89% @ | 60% @ | 22% @ | 60% @ |
| | 100 nM | 100 nM | 30 nM | 100 nM | 100 nM | 100 nM | 100 nM | 100 nM | 100 nM |

### Example 8: Mu-Opioid Receptor Activity Assays

The compound of Example 1 is tested in CHO-K1 cells expressing hOP3 (human mu-opioid receptor μ1 subtype) using an HTRF-based cAMP assay kit (cAMP Dynamic2 Assay Kit, from Cisbio, #62AM4PEB). Frozen cells are thawed in a 37° C. water bath and are resuspended in 10 mL of Ham's F-12 medium containing 10% FBS. Cells are recovered by centrifugation and resuspended in assay buffer (5 mM KCl, 1.25 mM MgSO₄, 124 mM NaCl, 25 mM HEPES, 13.3 mM glucose, 1.25 mM KH₂PO₄, 1.45 mM CaCl₂, 0.5 g/L protease-free BSA, supplemented with 1 mM IBMX). Buprenorphine, a mu-opioid receptor partial agonist, and naloxone, a mu-opioid receptor antagonist, and DAMGO, a synthetic opioid peptide full agonist, are run as controls.

For agonist assays, 12 μL of cell suspension (2500 cells/well) are mixed with 6 μL forskolin (10 μM final assay concentration), and 6 μL of the test compound at increasing concentrations are combined in the wells of a 384-well white plate and the plate is incubated for 30 minutes at room temperature. After addition of lysis buffer and one hour of further incubation, cAMP concentrations are measured according to the kit instructions. All assay points are determined in triplicate. Curve fitting is performed using XLfit software (IDBS) and EC₅₀ values are determined using a 4-parameter logistic fit. The agonist assay measures the ability of the test compound to inhibit forskolin-stimulated cAMP accumulation.

For antagonist assays, 12 μL of cell suspension (2500 cells/well) are mixed with 6 μL of the test compound at increasing concentrations, and combined in the wells of a 384-well white plate and the plate is incubated for 10 minutes at room temperature. 6 μL of a mixture of DAMGO (D-Ala²-N-MePhe⁴-Gly-ol-enkephelin, 10 nM final assay concentration) and forskolin (10 μM final assay concentration) are added, and the plates are incubated for 30 minutes at room temperature. After addition of lysis buffer, and one hour of further incubation, cAMP concentrations are measured according the kit instructions. All assay points are determined in triplicate. Curve fitting is performed using XLfit software (IDBS) and IC₅₀ values are determined using a 4-parameter logistic fit. Apparent dissociation constants (KB) are calculated using the modified Cheng-Prusoff equation. The antagonist assay measures the ability of the test compound to reverse the inhibition of forskolin-induced cAMP accumulation caused by DAMGO.

The results are shown in the Table below. The results demonstrate that the compound of Example 1 is a weak antagonist of the Mu receptor, showing much higher IC₅₀ compared to naloxone, and that it is a moderately high affinity, but partial agonist, showing only about 22% agonist activity relative to DAMGO (as compared to about 79% activity for buprenorphine relative to DAMGO). The compound of Example 1 is also shown to have moderately strong partial agonist activity.

| Compound | Antagonist IC₅₀ (nM) | Agonist EC₅₀ (nM) | KB (nM) |
|---|---|---|---|
| Naloxone | 5.80 | — | 0.65 |
| DAMGO | — | 1.56 | — |
| Buprenorphine | — | 0.95 | — |
| Cmpd. Ex. 1 | 641 | 64.5 | 71.4 |

Buprenorphine is a drug used for opioid withdrawal, but it suffers from the problem that users can become addicted due to its high partial agonist activity. To offset this, the commercial combination of buprenorphine with naloxone is used (sold as Suboxone). Without being bound by theory, it is believed that the compounds of the present invention, which are weaker partial Mu agonists than buprenorphine, with some moderate antagonistic activity, will allow a patient to be more effectively treated for opioid withdrawal with lower risks of addiction.

These results show that the compounds of the invention act as partial agonists of the GPCR-signaling pathway of mu-opioid receptors, but, that in the presence of a full agonist (DAMGO), these compounds effectively compete for receptor binding, and thus act as antagonists of the full agonist. In effect, this means that in the presence of an opioid drug, such as fentanyl or fentanyl analogs, the compounds of the invention will competitively bind to and displace these full agonists from the mu-opioid receptors. Therefore, in practice, these compounds are effective as antagonists for the purpose of reversing overdose of fentanyl and fentanyl analogs.

### Example 9: GPCR Beta-Arrestin Signaling Assay

The Compound of Example 1 is studied using a beta-arrestin assay. The assay monitors the activation of a selected G-protein coupled receptor (GPCR) in a homogenous, non-imaging assay format using proprietary technology based on beta-galactosidase as a functional reporter. This enzyme is split into two inactive complementary portions, termed EA and PK, expressed as fusion proteins in the cell. The EA portion is fused to beta-arrestin and the PK portion is fused to the GPCR of interest, the human mu-opioid receptor. When the GPCR is activated and beta-arrestin is recruited to the receptor, the two portions of the enzyme are complemented restoring enzymatic activity, which is detected via chemiluminescence reagents.

The proprietary cell lines are seeded in a volume of 20 μL in 384-cell microplates and incubated at 37° C. For agonist determination, cells are incubated with Compound of Example 1 to induce a response. Intermediate dilution of Compound stock is performed to generate 5× Compound in assay buffer. 5 μL of 5× Compound solution is added to cells and incubated at 37° C. for 1.5 to 3 hours. Vehicle concentration is 1%. For antagonist determination, cells are pre-incubated with Compound of Example 1 followed by agonist ([Met]-enkephalin) challenge at the EC80 concentration of the agonist. Intermediate dilution of Compound stock is performed to generate 5× Compound in assay buffer. 5 μL of 5× Compound solution is added to cells and incubated at 37° C. or room temperature for 0.5 hours. Vehicle concentration is 1%. 5 μL of 6× EC80 agonist in assay buffer is then added and the cells are incubated at 37° C. for 1.5 to 3 hours. In both formats, assay signal is generated by a single addition of 12.5-15 μL of proprietary detection reagent cocktail, followed by 1 hour incubation at room temperature. Microplates are then read for chemiluminescent signal detection. Data is analyzed using CBIS data analysis software suite (ChemInnovation, CA). Control dose response curves are generated using [Met]-enkephalin as the positive control for the agonist format, and using naloxone hydrochloride as the positive control for antagonist format.

The results are presented in the table below:

| | Beta-Arrestin | Beta-Arrestin |
|---|---|---|
| Compound | Antagonist IC₅₀ (nM) | Agonist EC₅₀ (nM) |
| Naloxone | 5.88 | — |
| [Met]-enkephalin | — | 83.7 |
| Cmpd. Ex. 1 | 189 | >10,000 |

These results demonstrate that the Compound of Example 1 does not stimulate beta-arrestin signaling via the mu-opioid receptor at concentrations up to 10 μM, but that it is an antagonist with an IC₅₀ of 189 nM. In contrast, the full opioid agonist [Met]-enkephalin stimulates beta-arrestin signaling with an EC₅₀ of 84 nM.

### Example 10: Alpha1-Adrenergic Receptor Activity

### Example 10a: Receptor Binding Assay

The Compound of Example 1 is tested in a human alpha1A adrenergic receptor antagonist radioligand assay. Standard procedures are followed according to Schwinn, D A et al., J. Biol. Chem. 265:8183-89 (1990). Human recombinant CHO cells are used for the assay. Assay incubation is conducted for 60 minutes at room temperature. The antagonist radioligand is [3H] prazosin and the non-specific control is epinephrine (0.1 mM). The Compound of Example 1 is used from a stock solution at 0.01M in DMSO.

It is found that the Compound of Formula 1 is an antagonist of the alpha1A adrenergic receptor with a binding Ki of 28 nM.

### Example 10b: Functional Assay

Further studies are conducted in a functional alpha-1A adrenergic receptor assay measuring intracellular calcium response using aequorin luminescence.

For the agonist assay, CHO-K₁ cells expressing human alpha1A-adrenergic receptor are suspended in Ham's F-12 medium containing 10% FBS. Cells are then recovered via centrifugation and resuspended in prewarmed assay buffer (DMEM/HAM's F12 w/HEPES) at 3×10⁵ cells/mL in a Falcon tube. Coelenterazine h is added to a final concentration of 5 μM, and the tube is wrapped in aluminum foil and placed on a rotating wheel for 4 hours at room temperature. Cells are then diluted 3× in assay buffer and transferred to a beaker wrapped in aluminum foil. After stirring 1 hour, 50 μL of cells (5,000 cells/well) are injected into 50 μL of the Compound of Example 1 at increasing concentrations in a 96-well plate. Light emission is immediately recorded for 20 seconds using a luminescence detector. Digitonin at 50 μM in assay buffer is used as a positive control to measure receptor-independent cellular calcium response. Phenylephrine is used as a positive control for receptor activity. Agonist activity is measured as the degree of light emission stimulated by the test compound.

For the antagonist assay, 50 μL of cells (5,000 cells/well) are mixed with 50 μL of the Compound of Example 1 at increasing concentrations in a 96-well plate, and incubated for 15 minutes at room temperature. Then, 50 μL of phenylephrine is added for a final concentration of 50 nM (corresponding to the EC80 of phenylephrine). Light emission is immediately recorded for 20 seconds using a luminescence detector. Antagonist activity is measured by the decrease in light emission produced by the phenylephrine. Tamsulosin is used as a positive control.

The results show that the compound of Formula 1 has no agonist activity at the receptor, but it is an antagonist with an IC₅₀ of 33 nM.

### Example 11: Competitive Inhibition of Fentanyl Binding

Using the procedures described in Example 10, the ability of the Compound of Example 1 to functionally inhibit fentanyl-induced beta-arrestin signaling is examined.

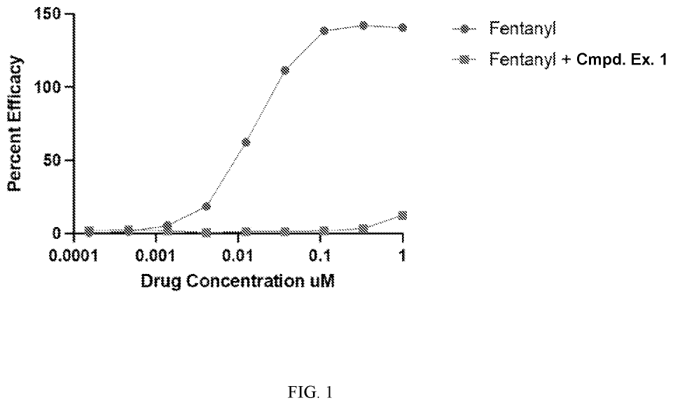

Following the agonist protocol, the functional activity of fentanyl is examined in the absence of and in the presence of 10 μM Compound of Example 1. The results are shown in the table below, and in FIG. 1.

| | Beta-Arrestin |
|---|---|
| Test Sample | Agonist EC₅₀ (nM) |
| [Met]-enkephalin | 338 |
| Fentanyl | 15 |
| Fentanyl + Cmpd. Ex. 1 | >1,000 |

The results demonstrate that the Compound of Example fully inhibits fentanyl-induced agonism of the mu-opioid receptor beta-arrestin signaling pathway.

### Example 12: Pharmacokinetics

The oral pharmacokinetics of the compound of Example 1 has been studied in cynomolgus monkeys using standard procedures. Oral dosing was performed using the compound of Example 1 in tosylate salt form at a dose of 2.8 mg/kg, formulated in PEG-400. Intravenous (IV) dosing was performed using the compound of Example 1 in tosylate salt form at a dose of 1 mg/kg in sterile water with 45% Trappsol (beta-cyclodextrin) and 1% DMSO. The results are shown in the following table:

| Parameter | Oral Dosing | IV dosing |
|---|---|---|
| Time to max. plasma conc. (Tmax) (hours) | 3 | |
| Max. plasma conc. (Cmax) (ng/mL | 114 | |
| Half-life (T_{1/2}) (hours) | 9.6 | 4.6 |
| AUC_{0-∞} (ng-h/mL) | 1408 | 1150 |
| Volume of distribution (L/kg) | | 6.2 |
| Clearance (L/h/kg) | | 0.9 |
| Oral Bioavailability | 43.7% | |

The compound of Formula I is also found to have human plasma protein binding of 91.6%.

### Example 13: Naloxone-Precipitated Oxycodone-Dependent Withdrawal Study in Mice

Oxycodone is administered to adult male C₅₇BL/6J mice for 8 days at an increasing dose regimen of 9, 17.8, 23.7, and 33 mg/kg twice per day (7 hours between injections) on days 1-2, 3-4, 5-6 and 7-8 respectively. On the morning of the ninth day, the mice are administered the compound of Example 1 at either 0.3, 1 or 3 mg/kg subcutaneous. This is followed 30 minute later by either an injection of vehicle or with an injection of 3 mg/kg of naloxone. Another cohort of mice serve as negative controls, and instead of oxycodone, these mice are administered saline on days 1 to 8. On day 9, these mice are administered either vehicle (followed by naloxone, as above) or the compound of Example 1 at 3 mg/kg, s.c. (followed by naloxone, as above).

On day 9, immediately after the injection of naloxone (or vehicle), the mice are individually placed in clear, plastic cages and are observed continuously for thirty minutes. The mice are monitored for common somatic signs of opioid withdrawal, including jumping, wet dog shakes, paw tremors, backing, ptosis, and diarrhea. All such behaviors are recorded as new incidences when separated by at least one second or when interrupted by normal behavior. Animal body weights are also recorded immediately before and 30 minutes after the naloxone (or vehicle) injections. Data is analyzed with ANOVA followed by the Tukey test for multiple comparisons, when appropriate. Significant level is established at p<0.05.

The results are shown in the Table below:

| Dosing: (1) on days 1-8, | Total | | | Body |
|---|---|---|---|---|
| (2) on day 9, followed by | Number | Paw | | Weight |
| (3) 30 minutes later | of Signs | Tremors | Jumps | Loss |
| (1) Saline; (2) Vehicle, | 2.2 | 0.87 | 0 | 0.5% |
| (3) Naloxone | | | | |
| (1) Saline; (2) Compound | 5.3 | 0.12 | 0 | 0.4% |
| 3.0 mg/kg, (3) Naloxone | | | | |
| (1) Oxycodone; (2) | 155.1 | 73.6 | 63.2 | 7.8% |
| Compound 3.0 mg/kg, | | | | |
| (3) Vehicle | | | | |
| (1) Oxycodone; (2) | 77.5 | 19.6 | 40.6 | 7.5% |
| Compound 0.3 mg/kg, | | | | |
| (3) Naloxone 3 mg/kg | | | | |
| (1) Oxycodone; (2) | 62.5 | 14.8 | 34.8 | 6.0% |
| Compound 1.0 mg/kg, | | | | |
| (3) Naloxone 3 mg/kg | | | | |
| (1) Oxycodone; (2) | 39.5 | 0.5 | 26.6 | 4.0% |
| Compound 3.0 mg/kg, | | | | |
| (3) Naloxone 3 mg/kg | | | | |

Total number of signs includes paw tremors, jumps, and wet dog shakes. In oxycodone-treated mice, it is found that naloxone elicits a significant number of total signs, paw tremors, jumps and body weight change (p≤0.0001 for each), indicating precipitated withdrawal. At all doses tested, the compound of Example 1 produces a significant decrease in total number of signs and paw tremors precipitated by naloxone. In addition, at 3.0 mg/kg, the compound also produces a significant decrease in jumps and attenuated body weight loss.

These results demonstrate that the compound of Example 1 dose-dependently reduces the signs and symptoms of opioid withdrawal after the sudden cessation of opioid administration in opioid-dependent rats, and prevents the signs and symptoms of opioid withdrawal induced by naloxone.

### Example 14: Lack of Precipitated Withdrawal from Oxycodone

In a similar study design as for Example 13, mice are chronically treated with oxycodone or saline were challenged with ITI-333 or vehicle (Veh) and observed for manifestation of somatic signs of withdrawal (including jumps, wet dog shakes, paw tremors, backing, ptosis and diarrhea).

Adult male C57Bl/6 mice (Jackson Labs, Bar Harbor, ME) are administered oxycodone as described in Example 13. On the morning of the ninth day, mice are administered oxycodone (33 mg/kg, s.c.) followed 2 hours later with an injection of the compound of Example 1 (3, 10, or 17.8 mg/kg, s.c.; n=8 each) or vehicle (n=8). A separate group of mice are chronically administered saline instead of oxycodone and are challenged with the compound of Example 1 (17.8 mg/kg, s.c.; n=8) or vehicle (n=8) on day 9 to evaluate the effects of the compound of Example 1 alone. Thirty minutes following vehicle or compound injections on day 9, the mice are individually placed in plastic cages and observed for somatic signs of withdrawal as described in Example 13. Data are analyzed with ANOVA followed by Tukey tests for multiple comparisons.

The results show that mice chronically administered oxycodone and given the compound of Example 1 (at doses <10 mg/kg) do not differ from mice administered vehicle on the number of paw tremors or jumps. The compound of Example 1 (<10 mg/kg) also does not induce further body weight loss in mice receiving oxycodone chronically. However, with escalating doses, the compound of Example 1 induced greater total withdrawal signs (p<0.0001). At 10 mg/kg, it induces significantly more total withdrawal signs compared with morphine alone (p<0.05), mainly attributable to increases in wet dog shakes. Following chronic treatment with saline, the compound of Example 1 does not produce any significant effects on somatic signs or loss of body weight relative to mice administered vehicle on Day 9 (p>0.05).

### Example 15: Reversal of Fentanyl-Induced Respiratory Depression in Rats

A study is conducted to determine the potential effects of the compound of Example 1, administered intravenously, on the respiratory depression induced by fentanyl in the conscious rat.

Animals are acclimated to their housing and the laboratory procedures over a minimum period of 5 days prior to initiation of dosing. Animals are selected by body weight and apparent good health and are randomly assigned to the study groups. Crl:CD rats from Charles River Laboratories weighing 150-255 grams at the initiation of dosing and aged 6-7 weeks are used in the study, and are divided into six groups. In Group (1), the negative control, animals are pre-treated with 3 mL/kg vehicle (s.c.) and then treated with 5 mL/kg vehicle (i.v.). In Group (2), the positive control, animals are pre-treated with 0.15 mg/kg fentanyl (s.c., 0.05 mg/mL) and then treated with vehicle (i.v.). In Group (3), the first test group, animals are pre-treated with 0.15 mg/kg fentanyl (s.c., 0.05 mg/mL), and then treated with 1 mg/kg of the compound of Example 1 (i.v.). In Group (4) and (5), the same protocol is administered as for Group (3), except using the higher doses of 3.0 mg/kg and 5.0 mg/kg of the compound of Example 1. In the final group, Group (6), animals are pre-treated with vehicle (i.v) and then treated with the compound of Example 1 at 5.0 mg/kg (i.v.). All intravenous treatment is administered by infusion over 5 minutes.

The animals are trained initially for two days immediately preceding the study, in a head-out plethysmograph chamber for approximately 10-15 minutes on each day. On the day of dosing, each animal is weighed and placed in the plethysmograph chamber and allowed to stabilize for at least 5 minutes. Following stabilization, respiratory parameters (respiratory rate, tidal volume and minute volume) are measured for 5 minutes continuously to obtain the pre-dose baseline values. The animal is then removed from the chamber and dosed as per group assignments. Following dosing, each animal is returned to its designated plethysmograph chamber and the respiratory parameters are measured at 5-minute intervals for 15 minutes. After each reading the animal is removed from the plethysmograph chamber. Prior to the next scheduled reading the animal is placed back in the plethysmograph chamber and allowed to stabilize for at least 5 minutes before another reading is taken. Respiratory data are acquired and analyzed using PONEMAH Physiology Platform (Ponemah v.5.20 pulmonary). Individual values of tidal volume and minute volume for the test article-treated groups are compared to vehicle control and baseline using unpaired T-tests.

The results are shown in the following table (TV: Tidal Volume; MV: Minute Volume):

| | N | Mean TV (mL) | Mean MV (mL) |
|---|---|---|---|
| Group (1) (neg. control) | 30 | 1.58 | 239 |
| Group (2) (pos. control) | 30 | 0.86 | 83 |
| Group (3) [1.0 mg/kg Ex. 1] | 5 | 1.12 | 112 |
| Group (4) [3.0 mg/kg Ex. 1] | 6 | 1.27 | 161 |
| Group (5) [5.0 mg/kg Ex. 1] | 6 | 1.48 | 163 |
| Group (6) [control Ex. 1] | 6 | 1.73 | 210 |

The result demonstrate that fentanyl rapidly induces respiratory depression, indicated by decreased minute volume (volume of air delivered over one minutes) and decreased tidal volume (volume of air delivered with each breath). The compound of Example 1 clearly prevents this respiratory depression, maintaining the animals at near-normal tidal volume and a slightly depressed minute volume at both higher doses, with partial effect at the lowest dose tested.

1. A method for one or more of the following:
(a) treating or reversing F/FA overdose;
(b) treating or reversing F/FA-induced respiratory depression;
(c) treating or reversing F/FA-induced muscle rigidity;
(d) treating or reversing F/A-induced laryngospasm;
(e) reversing or inhibiting binding of F/FA to mu-opioid receptors in the central nervous system (e.g., in the locus coeruleus);
(f) inhibiting F/FA-induced beta-arrestin signaling in the central nervous system (e.g., in the locus coeruleus);
(g) preventing death from F/FA overdose; and
(h) anesthetic recovery (e.g., following surgery);
the method comprising administering to a patient in need thereof an effective amount of a Compound of Formula I: 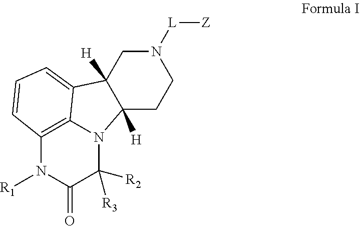
R¹ is H, C₁₋₆alkyl, —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸;
R² and R³ are independently selected from H, D, C₁₋₆alkyl (e.g., methyl), C₁₋₆alkoxy (e.g., methoxy), halo (e.g., F), cyano, or hydroxy;
L is C₁₋₆alkylene (e.g., ethylene, propylene, or butylene), C₁₋₆alkoxy (e.g., propoxy or butoxy), C₂₋₃alkoxyC₁₋₃alkylene (e.g., —CH₂CH₂OCH₂—), C₁₋₆alkylamino or N—C₁₋₆alkyl C₁₋₆alkylamino (e.g., propylamino or N-methylpropylamino), C₁₋₆alkylthio (e.g., —CH₂CH₂CH₂S—), C₁₋₆alkylsulfonyl (e.g., —CH₂CH₂CH₂S(O)₂—), each of which is optionally substituted with one or more R⁴ moieties;
each R⁴ is independently selected from C₁₋₆alkyl (e.g., methyl), C₁₋₆alkoxy (e.g., methoxy), halo (e.g., F), cyano, or hydroxy;
Z is selected from aryl (e.g., phenyl) and heteroaryl (e.g., pyridyl, indazolyl, benzimidazolyl, benzisoxazolyl), wherein said aryl or heteroaryl is optionally substituted with one or more R⁴ moieties;
R⁸ is —C(R^{a})(R^{b})(R^{c}), —O—C(R^{a})(R^{b})(R^{c}), or —N(R^{d})(R^{e});
R^{a}, R^{b} and R^{c} are each independently selected from H and C₁₋₂₄alkyl;
R^{d} and R^{e} are each independently selected from H and C₁₋₂₄alkyl;
R⁶ and R⁷ are each independently selected from H, C₁₋₆alkyl, carboxy and C₁₋₆alkoxycarbonyl;
in free or salt form (e.g., pharmaceutically acceptable salt form), for example in an isolated or purified free or salt form (e.g., pharmaceutically acceptable salt form).

2. The method according to claim 1, wherein in the compound of Formula I, R¹ is H.

3. The method according to claim 1, wherein in the compound of Formula I, wherein R¹ is —C(O)—O—C(R^{a})(R^{b})(R^{c}), —C(O)—O—CH₂—O—C(R^{a})(R^{b})(R^{c}) or —C(R⁶)(R⁷)—O—C(O)—R⁸.

4. The method according to claim 1, wherein in the compound of Formula I, L is C₁₋₆alkylene (e.g., ethylene, propylene, or butylene) or C₁₋₆alkyoxy (e.g., propoxy or butoxy), each optionally substituted with one or more R⁴ moieties.

5. The method according to claim 1, wherein in the compound of Formula I, R² and R³ are each H.

6. The method according to claim 1, wherein in the compound of Formula I, Z is phenyl substituted with one fluoro (e.g., 2-fluorophenyl, 3-fluorophenyl or 4-fluorophenyl).

7. The method according to claim 1, wherein in the compound of Formula I, Z is heteroaryl (e.g., pyridyl, indazolyl, benzimidazolyl, benzisoxazolyl), optionally substituted with one or more R⁴ moieties.

8. The method according to claim 1, wherein the compound of Formula I is selected from the group consisting of: 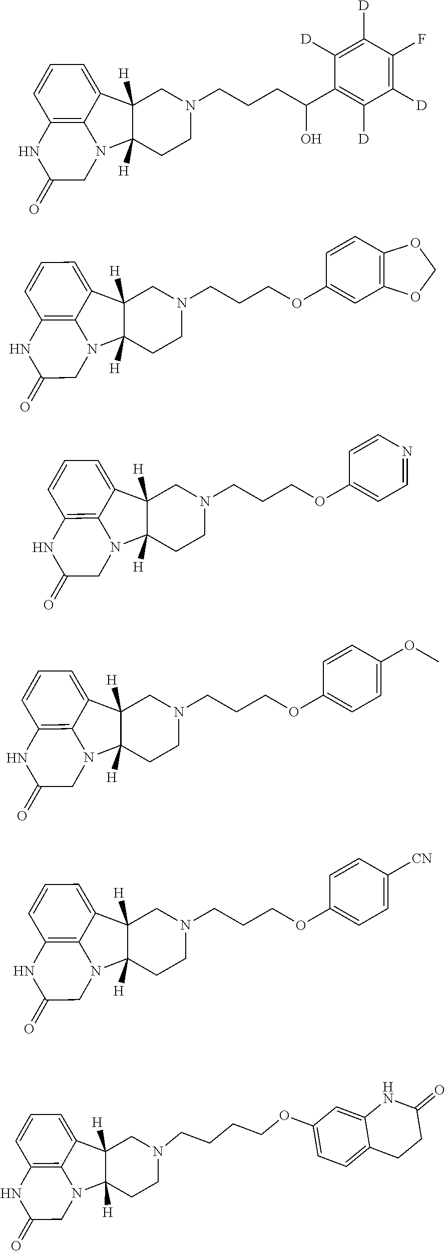 [SMILES: [2H]c1c([2H])c(C(O)CCCN2CC[C@H]3[C@@H](C2)c2cccc4c2N3CC(=O)N4)c([2H])c([2H])c1F ; O=C1CN2c3c(cccc3[C@@H]3CN(CCCOc4ccc5c(c4)OCO5)CC[C@@H]32)N1 ; O=C1CN2c3c(cccc3[C@@H]3CN(CCCOc4ccncc4)CC[C@@H]32)N1 ; COc1ccc(OCCCN2CC[C@H]3[C@@H](C2)c2cccc4c2N3CC(=O)N4)cc1 ; N#Cc1ccc(OCCCN2CC[C@H]3[C@@H](C2)c2cccc4c2N3CC(=O)N4)cc1 ; O=C1CCc2ccc(OCCCCN3CC[C@H]4[C@@H](C3)c3cccc5c3N4CC(=O)N5)cc2N1] 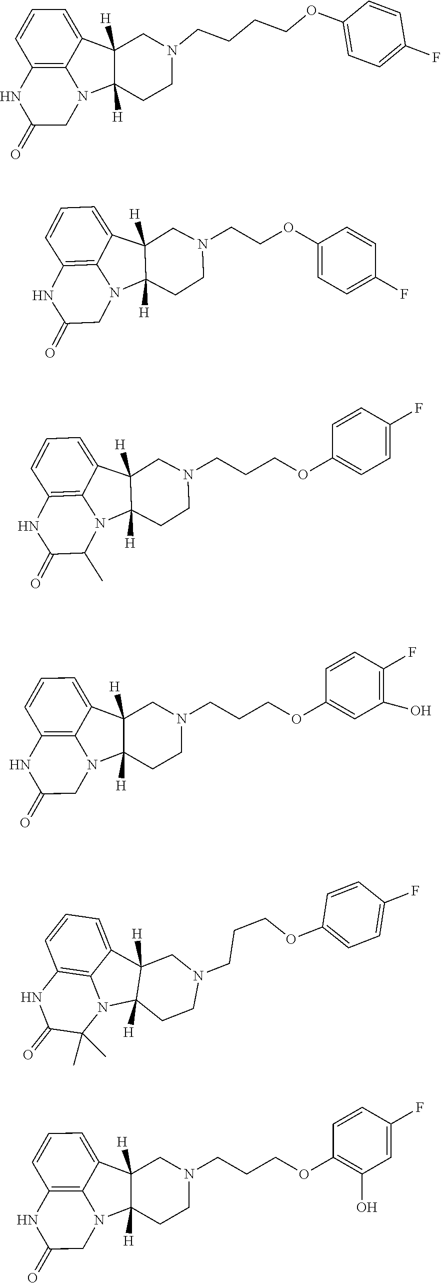 [SMILES: O=C1CN2c3c(cccc3[C@@H]3CN(CCCCOc4ccc(F)cc4)CC[C@@H]32)N1 ; O=C1CN2c3c(cccc3[C@@H]3CN(CCOc4ccc(F)cc4)CC[C@@H]32)N1 ; CC1C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)c(O)c4)CC[C@@H]32)N1 ; CC1(C)C(=O)Nc2cccc3c2N1[C@H]1CCN(CCCOc2ccc(F)cc2)C[C@@H]31 ; O=C1CN2c3c(cccc3[C@@H]3CN(CCCOc4ccc(F)cc4O)CC[C@@H]32)N1] 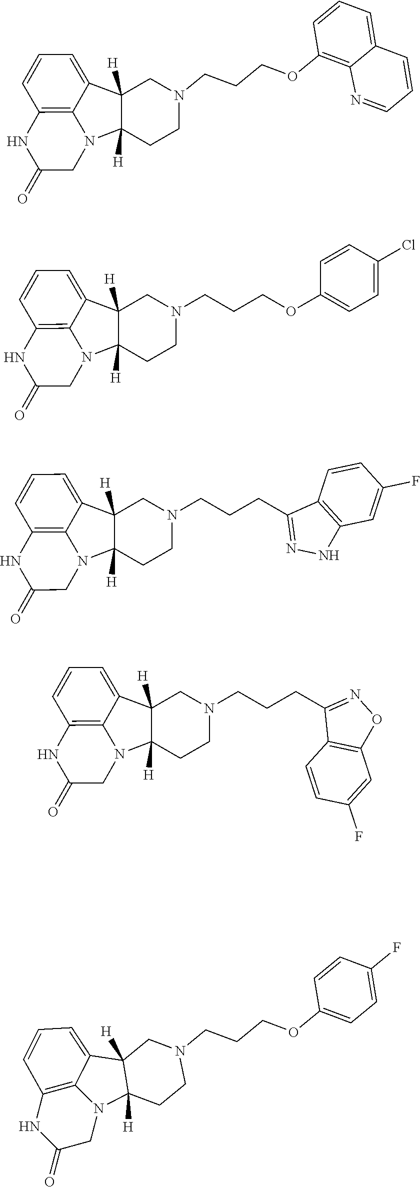 [SMILES: O=C1CN2c3c(cccc3[C@@H]3CN(CCCOc4cccc5cccnc45)CC[C@@H]32)N1 ; O=C1CN2c3c(cccc3[C@@H]3CN(CCCOc4ccc(Cl)cc4)CC[C@@H]32)N1 ; O=C1CN2c3c(cccc3[C@@H]3CN(CCCc4n[nH]c5cc(F)ccc45)CC[C@@H]32)N1 ; O=C1CN2c3c(cccc3[C@@H]3CN(CCCc4noc5cc(F)ccc45)CC[C@@H]32)N1 ; O=C1CN2c3c(cccc3[C@@H]3CN(CCCOc4ccc(F)cc4)CC[C@@H]32)N1]
each independently in free or pharmaceutically acceptable salt form.

9. The method according to claim 1, wherein the compound of Formula I is selected from the group consisting of: 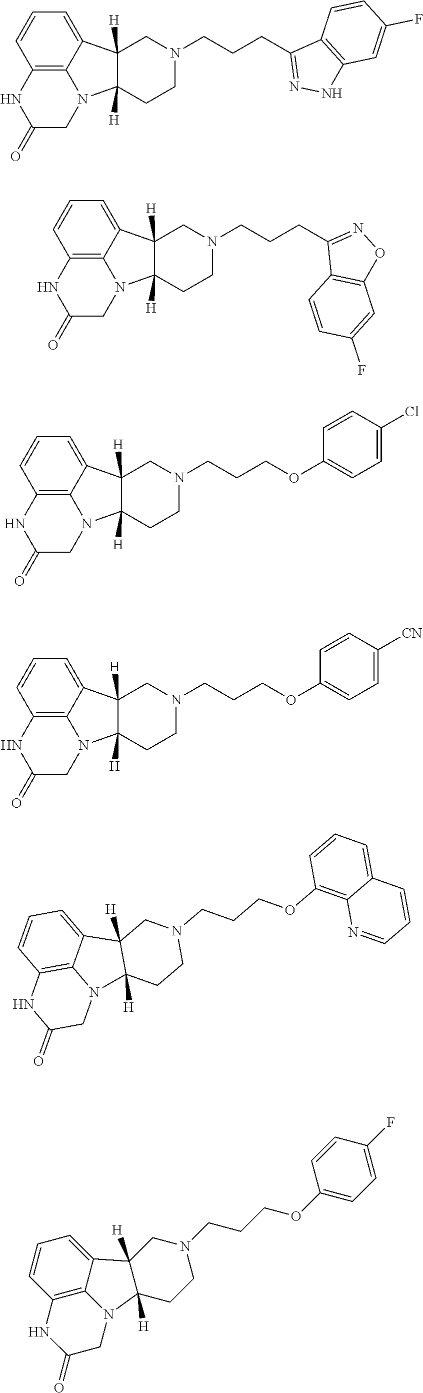
each independently in free or pharmaceutically acceptable salt form.

10. The method according to claim 1, wherein the compound of Formula I is: 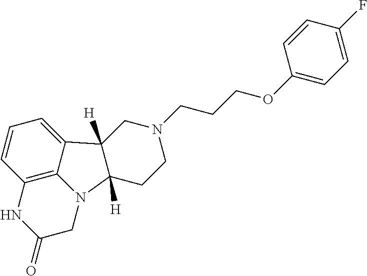 [SMILES: O=C1CN2c3c(cccc3[C@@H]3CN(CCCOc4ccc(F)cc4)CC[C@@H]32)N1]
in free or pharmaceutically acceptable salt form.

11. The method according to claim 1, wherein the compound of Formula I is in salt form, e.g., in the form of a pharmaceutically acceptable salt.

12. The method according to claim 1, wherein the compound of Formula I is administered in the form of a pharmaceutical composition comprising the compound of Formula I in admixture with a pharmaceutically acceptable diluent or carrier.

13. The method according to claim 12, wherein the pharmaceutical composition is formulated for single dose administration (e.g., tablets, capsules, wafers, single-use injections, single-use ampules or vials for intranasal administration, single use ampules or vials for injection, single use intranasal sprays).

14. The method according to claim 12, wherein the pharmaceutical composition is formulated for intranasal or intrapulmonary administration (e.g., as an aerosol, mist, or powder for inhalation).

15. The method according to claim 12, wherein the pharmaceutical composition is formulated for administration by injection, for example, as a sterile aqueous solution, e.g., for intravenous, subcutaneous, or intramuscular injection.

16. The method according to claim 15, wherein the pharmaceutical composition is formulated for and/or packaged as a pre-filled syringe for injection, as an auto-injector, or as a sterile solution in a vial for injection or intranasal administration.

17. The method according to claim 1, wherein the patient demonstrates chest wall rigidity.

18. The method according to claim 1, wherein the patient demonstrates laryngospasm.

19. The method according to claim 1, wherein the patient is diagnosed with or suspected or having wooden chest syndrome (WCS), fentanyl-induced muscle rigidity (FIMR) or fentanyl-induced respiratory muscle rigidity (FIRMR).

20. The method according to claim 1, wherein the patient has not responded to, or has not responded adequately to (e.g., with respect to signs or symptoms of respiratory depression) a single dose or multiple doses of a mu-opioid antagonist (e.g., 0.1 to 4 mg) administered by any route (e.g., intranasal, intravenous, subcutaneous, or intramuscular), such as naloxone.

21. The method according to claim 1, wherein the effective amount of the Compound of Formula I is an amount effective to reverse one or more of: respiratory arrest, respiratory depression, skeletal muscle spasm, chest wall rigidity, laryngospasm, pupillary constriction, cardiac arrest, bradycardia, or unconsciousness.

22. The method according to claim 1, wherein the F/FA is selected from fentanyl, sufentanil, alfentanil, remifentanil, carfentanil, thiafentanil, lofentanil, ocfentanil, trefantinil, and brifentanil.

23. The method according to claim 1, wherein the method does not cause precipitated withdrawal in the patient, e.g., withdrawal symptoms selected from tachycardia, nausea, vomiting, diarrhea, extreme anxiety, restless legs, muscle aches, and profuse sweating.

24. The method according to claim 1, wherein the source of the F/FA is another illicit drug which is adulterated with the F/FA, such as cocaine, amphetamine, methamphetamine, or marijuana.
